(19) United States
(12) Patent Application Publication
(10) Pub. No.: US 2025/0000964 A1
(43) Pub. Date: Jan. 2, 2025

(54) NEOADJUVANT USAGE OF PLANT VIRUS OR VIRUS-LIKE PARTICLES FOR CANCER TREATMENT

(71) Applicant: TRUSTEES OF DARTMOUTH COLLEGE, Hanover, NH (US)

(72) Inventors: Steven Fiering, Hanover, NH (US); Hugo Arias-Pulido, Hanover, NH (US); Laura Pena, Madrid (ES); Nicole F. Steinmetz, San Diego, CA (US); Gregory W. Ho, Hanover, NH (US)

(21) Appl. No.: 18/703,610

(22) PCT Filed: Oct. 24, 2022
(86) PCT No.: PCT/US2022/078599

§ 371 Date: Apr. 22, 2024

Related U.S. Application Data
(60) Provisional application No. 63/270,679, filed on Oct. 22, 2021.

(51) Int. Cl. A61K 39/12; A61P 35/00; C07K 16/28; C12N 7/00

(52) U.S. Cl. CPC A61K 39/12; A61P 35/00; C07K 16/2818; C07K 16/2827; C12N 7/00; C12N 2770/18034

(57) ABSTRACT

A neoadjuvant for use in treating cancer includes an in situ vaccine and optionally an immune check point therapeutic. The in situ vaccine includes at least one of cowpea mosaic virus or cowpea mosaic virus-like particles.

### RELATED APPLICATION

This application claims priority from U.S. Provisional Application No. 63/270,679, filed Oct. 22, 2021, the subject matter of which are incorporated herein by reference in their entirety.

### GOVERNMENT FUNDING

This invention was made with government support under CA218292 and CA224605 awarded by the National Science Foundation. The government has certain rights in the invention.

### BACKGROUND

Inflammatory breast cancer (IBC) is a rare, aggressive, and highly metastatic form of human breast cancer (BC). At time of diagnosis, all IBC patients have lymph node involvement and about 40% have distant identifiable metastases. IBC accounts for roughly 2.5% of all newly diagnosed BC in the US, but is responsible for about 10% of BC-related deaths, making it the deadliest form of BC. Anthracycline-taxane-based chemotherapy, with targeted therapy when appropriate, remains the backbone of neoadjuvant therapy for IBC; and its efficacy is minimally effective as shown by a 15-year survival rate of about 20%-30%. The inability to identify effective therapy has multiple factors, but two major contributing factors are the absence of optimal models to identify mechanisms involved in the aggressive behavior of IBC, and the associated lack of knowledge of in vivo IBC biology. The limited knowledge we do have has been obtained from in vitro studies using cell lines and patient-derived xenograft models using immunodeficient mice. While cell lines are useful for drug sensitivity studies, and published data reveal a remarkable correlation between drug responses in patient-derived xenograft models and clinical patient outcomes, a direct extrapolation of useful data obtained from these models into human clinical trials is not always possible. This is particularly true for immunotherapy studies since human xenografts can only be studied in vivo using immunodeficient mice.

Rodent cancer immunotherapy studies usually use syngeneic cell lines in inbred mice, but there is no rodent cell line model of IBC or canine inflammatory mammary cancer (IMC). Canine cancer patients, however, can bridge rodent lab studies and human clinical trials. Cancers in dogs occur spontaneously, have clinical and pathophysiological presentation equivalent to human cancers, and share genomic and immune features. Canine patients are outbred, have intact immune systems and a tumor that, like spontaneous human tumors, is predominantly ‘self’ immunologically, making them a uniquely valuable model for immunotherapy studies. The progression of disease in dogs recapitulates disease course in humans to a much greater extent than rodent cancer models. Canine mammary tumors are the most frequent neoplasia in sexually intact female dogs. The annual incidence of mammary tumors within all canine tumors is about 17%, and the frequency of malignant tumors varies from 48% to 70% of the overall mammary tumor patients. Strikingly, dogs are also affected by IMC which, as in human IBC, is the most aggressive type of mammary tumors, with a reported incidence of 18% of all canine malignant mammary tumors. IMC dogs live an average of 1 month post diagnosis in absence of specific treatment. As we have recently reviewed, canine IMC represents a unique and excellent therapeutic model to evaluate the clinical efficacy of new anticancer agents, approaches or combinations, including immunotherapy.

### SUMMARY

Embodiments described herein relate to the use of an in situ vaccine that includes plant virus or plant virus-like particles as a neoadjuvant for the treatment of cancer, such as inflammatory breast cancer or inflammatory mammary cancer, in a subject in need thereof. We found that an in situ vaccine that includes plant virus or plant virus-like particles, such as cowpea mosaic virus or virus-like particles, when administered as a neoadjuvant directly or locally to cancer or tumor, such as inflammatory breast or mammary cancer, that is inoperable or unresectable can reduce the cancer or tumor burden in a subject such that the cancer or tumor can be removed or reduced by surgical resection and/or further reduced by radiotherapy. Advantageously, an in situ vaccine administered as a neoadjuvant without the administration or use of a chemotherapeutic can decrease or reduce cancer or tumor burden without adverse events, such as cardiotoxicity, and provide an improved quality of life to cancer patients.

In some embodiments, a method of treating cancer in a subject in need thereof can include administering to the subject a neoadjuvant therapy that includes an in situ vaccine and optionally an immune check point therapy prior to surgical resection and/or radiotherapy of the cancer. The in situ vaccine can include at least one of cowpea mosaic virus or cowpea mosaic virus-like particles. The cancer can be treated by surgical resection and/or radiotherapy following administration of the neoadjuvant therapy. Optionally, an adjuvant therapy can be administered to the subject after surgical resection and/or radiotherapy of the cancer.

In some embodiments, the cancer can be a locally advanced, metastatic, and/or inflammatory cancer.

In other embodiments, the cancer can be inflammatory breast cancer, such as stage III or stage IV inflammatory breast cancer or PD-L1+ inflammatory breast cancer.

In some embodiments, the neoadjuvant can be administered without or in the absence of chemotherapy.

In other embodiments, the immune check point therapy can include

administration of an immune check point inhibitor to the subject. For example, the immune check point therapy can include administration of at least one of an anti-PD-1 antibody or an anti-PD-L1 antibody to the subject.

In some embodiments, the adjuvant therapy can include at least one of radiotherapy, immunotherapy, chemotherapy, ultrasound therapy, or hormone therapy.

In some embodiments, the neoadjuvant therapy can be administered at an amount effective to reduce cancer burden in the subject.

In other embodiments, the neoadjuvant therapy can be administered at an amount effective to decrease Treg/CD8+ ratio and/or increase CD8+GZMB+ T cell level in blood of the subject.

In some embodiments, the cancer is inflammatory breast cancer, which is inoperable prior to administration of the neoadjuvant therapy and optional immune check point therapy, and the neoadjuvant therapy and optional immune checkpoint therapy is administered at amount effective for the inflammatory breast cancer to be operable by surgical resection.

In other embodiments, the cowpea mosaic virus and cowpea mosaic virus-like are administered by injection into the cancer.

Other embodiments described herein relate to a method of treating breast or mammary cancer in a subject in need thereof. The method includes administering to a subject with locally advanced or inflammatory breast or mammary cancer a neoadjuvant therapy that includes an in situ vaccine and optionally an immune check point therapy prior to surgical resection of the breast or mammary cancer. The in situ vaccine can include at least one of cowpea mosaic virus or cowpea mosaic virus-like particles. Following administration of the neoadjuvant, the breast or mammary cancer can be removed by surgical resection. Optionally an adjuvant therapy can be administered to the subject after surgical resection of the breast or mammary cancer.

In some embodiments, the breast cancer is inflammatory breast cancer, such as stage III or stage IV inflammatory breast cancer or PD-L1+ inflammatory breast cancer.

In some embodiments, the neoadjuvant can be administered without or in the absence of chemotherapy.

In some embodiments, the immune check point therapy includes administration of an immune check point inhibitor to the subject. The immune check point therapy can include systemic administration of at least one of an anti-PD-1 antibody or an anti-PD-L1 antibody to the subject.

In some embodiments, the adjuvant therapy can include at least one of radiotherapy, immunotherapy, chemotherapy, ultrasound therapy, or hormone therapy.

In some embodiments, the neoadjuvant therapy can be administered in at least one dose effective to reduce cancer burden in the subject.

In other embodiments, the neoadjuvant therapy can be administered in an amount effective to decrease Treg/CD8+ ratio and/or increase CD8+GZMB+ T cell level in blood of the subject.

In some embodiments, the inflammatory breast cancer is inoperable prior to administration of the neoadjuvant therapy and the neoadjuvant therapy is administered at amount effective for the inflammatory breast cancer to be operable by surgical resection.

In some embodiments, the cowpea mosaic virus and cowpea mosaic virus-like particles are administered by direct injection into the breast cancer.

Still other embodiments, relate to a neoadjuvant for use in treating cancer. The neoadjuvant can include an in situ vaccine and optionally an immune check point therapeutic.

The in situ vaccine includes at least one of cowpea mosaic virus or cowpea mosaic virus-like particles.

In some embodiments, the neoadjuvant therapy is free of a chemotherapeutic.

In other embodiments, the in situ vaccine and optional immune checkpoint therapeutic are provided at an amount effective to reduce cancer burden in a subject.

In some embodiments, the cancer prior to administration of the neoadjuvant is inoperable and after administration to the cancer operable by surgical resection.

In some embodiments, the in situ vaccine is formulated for direct injection into the cancer.

In other embodiments, the immune check point therapeutic is formulated for systemic administration to the subject.

In some embodiments, the immune check point therapeutic includes an immune check point inhibitor.

In some embodiments, the immune check point therapeutic includes at least one of an anti-PD-1 antibody or an anti-PD-L1 antibody.

In other embodiments, the cancer is inflammatory breast or mammary cancer, such as stage III or stage IV inflammatory breast cancer or PD-L1+ inflammatory breast cancer.

### BRIEF DESCRIPTION OF THE DRAWINGS

FIGS. 1(A-D) illustrate neoadjuvant in situ eCPMV immunotherapy induced tumor regression in canine IMC patients. IMC patients were treated and followed up as described in the Methods. (A, B) Tumor growth inhibition (TGI) as percentage of growth relative to D0. (A) % TGI in control canine IMC patients treated with medical therapy from D0, but no eCPMV therapy. Tumor volume changes in P7 during follow-up were not available. (B) % TGI in eCPMV-treated IMC patients. Dx refers to measurements done at D7 (P1, P2, P3, P4) and D9 (P5). Dy refers to measurements at D14 (P2, P3); D15 (P4), D17 (P5) and D19 (P1). Black arrows indicate eCPMV immunotherapy for all dogs; blue, for P1, P2 and P4. (C, D) Tumor growth kinetics in P1 (C) until surgery at D92 indicated by a red arrowhead, and P2 (D) until last day of follow-up at D79. Large black arrow in C indicates start of anti-COX2 therapy (15 days before eCPMV immunotherapy). Black arrows in C indicate eCPMV immunotherapy given to T1 and *, medical therapy as described in the Methods section. P1 and P2 patients have a second tumor mass (black broken line) (C, D). Blue arrows indicates treatment provided to the largest (T1; solid red line) and smallest (T2; interrupted black line) tumors in P1 (C) and both tumors in P2 (D). eCPMV, empty cowpea mosaic virus; IMC, inflammatory mammary cancer.

FIGS. 2(A-C) illustrate neoadjuvant in situ eCPMV immunotherapy induced changes in T cell populations in canine IMC patients. PBMCs were processed for flow analysis as described in Methods. (A) Change of Treg⁺/CD8⁺ ratio in IMC patients before (D0) and at first isolation after eCPMV treatment (Dx). Asterisk (*) indicates p<0.05 in a paired t-test. (B) Individual changes in Treg⁺/CD8⁺ ratio in IMC patients treated with eCPMV. Black arrows indicate eCPMV therapy for all dogs; blue for P1, P2, and P4; red for P1, and brown for P2. (C) Percentage change in CD8⁺GZMB⁺ T cell population during eCPMV treatment; values are represented as percentage change over D0. Arrowheads in the x-axis indicate surgery time on D17 for P5 and D92 for P1. eCPMV, empty cowpea mosaic virus; IMC, inflammatory mammary cancer; PBMCs, peripheral blood mononuclear cells.

FIG. 3 illustrates neoadjuvant in situ eCPMV immunotherapy induced a high neutrophilic infiltration in tumor samples and tumor emboli. Representative histopathology and immunostaining of tumor tissues from pretreatment/control and post-treatment samples. A strong neutrophilic infiltration is seen in post-treatment tumor tissues and emboli than in pretreatment tissues and tumor emboli as indicated by H&E and myeloperoxidase (MPO) expression. Higher Ki-67 proliferation index is observed in pretreatment tumor tissues and emboli than in post-treatment tumor tissues and emboli. There is no difference in cleaved caspase 3 (CC-3) immunolabeling between pretreatment tumor tissues and emboli than in post-treatment tumor tissues and emboli. eCPMV, empty cowpea mosaic virus.

FIG. 4 illustrates neoadjuvant in situ eCPMV immunotherapy is associated with improved survival in canine IMC patients. Canine patients treated with eCPMV injections (continuous line) showed longer survival than dogs treated only with medical therapy (broken line), but not eCPMV therapy. Y-axis denotes the survival probability and x-axis the number of days after first treatment with eCPMV therapy. Kaplan-Meier analysis was performed as described in Materials and Methods. eCPMV, empty cowpea mosaic virus; IMC, inflammatory mammary cancer; SOC, standard of care.

FIG. 5 illustrates in situ eCPMV injections the eCPMV nanoparticles were diluted in 0.5 ml of sterile phosphate buffered saline (PBS) and injected using an insulin syringe (25 G needle without dead volume). The injected PBS volume was equally distributed in 3 to 5 locations within the injected tumor to perfuse the tumor with the nanoparticles as much as possible. For every eCPMV injection, the needle was inserted as illustrated above and the nanoparticles were slowly injected while the needle was slowly retracted. Pressure was put on the injection site before taking the needle out to avoid leakage.

FIGS. 6(A-B) illustrate neoadjuvant in situ eCPMV immunotherapy does not affect red blood cell and hemoglobin levels in treated canine IMC patients. Changes induced by eCPMV injections in hematocrit (A) and hemoglobin (B). Black circles indicate basal levels at D0, blue rectangles, at D7, and brown triangles at D14. NR, refers to normal range values.

FIGS. 7(A-E) illustrate neoadjuvant in situ eCPMV immunotherapy does not affect hepatic, renal and digestive functions in the vaccinated canine IMC patients. Changes induced by eCPMV injections in (A) protein (albumin and globulins), (B) glucose, (C) creatinine, (D) urea, and (E) ALT levels. Black circles indicate basal levels at D0, blue rectangles, at D7, and brown triangles at D14. NR, refers to normal range values.

FIGS. 8(A-D) illustrate neoadjuvant in situ eCPMV immunotherapy induced minimal changes in peripheral blood immune cells. Changes induced by eCPMV injections in lymphocytes (A), monocytes (B), mature neutrophils (C), and immature neutrophils (D). Black circles indicate basal levels at D0, blue rectangles, at D7, and brown triangles at D14. NR, refers to normal range values.

FIGS. 9(A-I) illustrate gating strategy for immunophenotyping of canine PBMCs. (A) Leukocytes (SSC-A/FSC-A) were further defined as single (doublet exclusion FSC-H/FSC-A) live cells (SSC-A/Viability Aqua). (B) Live leukocytes were further discriminated in CD45⁺ leukocytes and CD14⁺CD45⁺ monocytes. (C) Differential expression of MHCII and CD4 on monocytic population. (D) CD45⁺ leukocytes were divided into lymphocytes (CD22⁺ B cells and CD5⁺ T cells) and (E) a CD22-CD5-cell population further characterized into MHCII+ antigen presenting cells and CD4⁺ neutrophils ([8] and references therein). (F) Gating for GzmB⁺CD3-NK cells. (G) Identification of CD4⁺ T helper and CD8⁺ T cytotoxic cells within the CD5⁺ T cell population, and FoxP3⁺ regulatory T cells differentially expressing CD25 within the CD4⁺ T cell population (H). (I) Expression of cytotoxic cell marker GzmB within the CD8⁺ T cell population. Parent population is indicated above the plots.

FIG. 10 illustrates neoadjuvant in situ eCPMV immunotherapy induced a differential effect on IL-8 in canine IMC patients. eCPMV injections induced an increase in IL-8 in P1 during the 14 day treatment, and a transient increase in P3, P4, and P5 by D7 with a subsequent decrease by D14. IL-8 levels in P1 were significantly higher at D14 compared to day 0 (P=0.039; linear regression analysis). Black circles indicate basal levels at D0, blue rectangles, at D7, and brown triangles at D14.

FIG. 11 illustrates neoadjuvant in situ eCPMV immunotherapy induces T lymphocyte infiltration and depletion of T regulatory lymphocytes in tumor samples. Representative immunostaining of tumor tissues from pre-treatment and post-treatment (P1) samples. A strong infiltration of T lymphocytes (CD3⁺) is observed in post-treatment tumor tissue compared to pre-treatment tissues. eCPMV treatment markedly reduces T regulatory lymphocytes (FoxP3⁺) in post-treatment tumor tissue.

FIG. 12 illustrates transmission electron microscopy images of negatively stained eCPMV at 49,000× enlargement. White bar=50 nm.

### DETAILED DESCRIPTION

### Definitions

As used in the description of the invention and the appended claims, the singular forms “a,” “an,” and “the” are intended to include the plural forms as well, unless the context clearly indicates otherwise. In addition, the recitations of numerical ranges by endpoints include all numbers subsumed within that range (e.g., 1 to 5 includes 1, 1.5, 2, 2.75, 3, 3.80, 4, 5, etc.).

The terms “comprise,” “comprising,” “include,” “including,” and “includes” when used in this specification and claims are intended to specify the presence of stated features, integers, components, or steps, but they do not preclude the presence or addition of one or more other features, integers, components, steps, or groups thereof. The terms “such as”, “e.g.,”, as used herein are non-limiting and are for illustrative purposes only. “Including” and “including but not limited to” are used interchangeably.

The term “or” as used herein should be understood to mean “and/or”, unless the context clearly indicates otherwise.

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys and liver.

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

The term “early stage breast cancer (EBC)” or “early breast cancer” is used herein to refer to breast cancer that has not spread beyond the breast or the axillary lymph nodes. This includes ductal carcinoma in situ and stage I, stage IIA, stage IIB, and stage IIIA breast cancers.

Reference to a tumor or cancer as a “Stage 0,” “Stage I,” “Stage II,” “Stage III,” or “Stage IV”, and various sub-stages within this classification, indicates classification of the tumor or cancer using the Overall Stage Grouping or Roman Numeral Staging methods known in the art. Although the actual stage of the cancer is dependent on the type of cancer, in general, a Stage 0 cancer is an in situ lesion, a Stage I cancer is small, localized tumor, a Stage II and III cancer is a local advanced tumor which exhibits involvement of the local lymph nodes, and a Stage IV cancer represents metastatic cancer. The specific stages for each type of tumor is known to the skilled clinician.

The term “metastatic breast cancer” means the state of breast cancer where the cancer cells are transmitted from the original site to one or more sites elsewhere in the body, by the blood vessels or lymphatics, to form one or more secondary tumors in one or more organs besides the breast.

An “advanced” cancer is one which has spread outside the site or organ of origin, either by local invasion or metastasis. Accordingly, the term “advanced” cancer includes both locally advanced and metastatic disease.

A “refractory” cancer is one which progresses even though an anti-tumor agent, such as a chemotherapy, is being administered to the cancer patient. An example of a refractory cancer is one which is platinum refractory.

A “recurrent” cancer is one which has regrown, either at the initial site or at a distant site, after a response to initial therapy, such as surgery.

A “locally recurrent” cancer is cancer that returns after treatment in the same place as a previously treated cancer.

An “operable” or “resectable” cancer is cancer which is confined to the primary organ and suitable for surgery (resection).

A “non-resectable” or “unresectable” cancer is not able to be removed (resected) by surgery.

“Neoadjuvant therapy” or “preoperative therapy” herein refers to therapy given prior to surgery. The goal of neoadjuvant therapy is to provide immediate systemic treatment, potentially eradicating micrometastases that would otherwise proliferate if the standard sequence of surgery followed by systemic therapy were followed. Neoadjuvant therapy may also help to reduce tumor size thereby allowing complete resection of initially unresectable tumors or preserving portions of the organ and its functions. Furthermore, neoadjuvant therapy permits an in vivo assessment of drug efficacy, which may guide the choice of subsequent treatments.

“Adjuvant therapy” herein refers to therapy given after definitive surgery, where no evidence of residual disease can be detected, so as to reduce the risk of disease recurrence. The goal of adjuvant therapy is to prevent recurrence of the cancer, and therefore to reduce the chance of cancer-related death. Adjuvant therapy herein specifically excludes neoadjuvant therapy.

“Definitive surgery” is used as that term is used within the medical community. Definitive surgery includes, for example, procedures, surgical or otherwise, that result in removal or resection of the tumor, including those that result in the removal or resection of all grossly visible tumor. Definitive surgery includes, for example, complete or curative resection or complete gross resection of the tumor. Definitive surgery includes procedures that occur in one or more stages, and includes, for example, multi-stage surgical procedures where one or more surgical or other procedures are performed prior to resection of the tumor. Definitive surgery includes procedures to remove or resect the tumor including involved organs, parts of organs and tissues, as well as surrounding organs, such as lymph nodes, parts of organs, or tissues. Removal may be incomplete such that tumor cells might remain even though undetected.

“Survival” refers to the patient remaining alive, and includes disease free survival (DFS), progression free survival (PFS) and overall survival (OS). Survival can be estimated by the Kaplan-Meier method, and any differences in survival are computed using the stratified log-rank test.

“Progression-Free Survival” (PFS) is the time from the first day of treatment to documented disease progression (including isolated CNS progression) or death from any cause on study, whichever occurs first.

“Disease free survival (DFS)” refers to the patient remaining alive, without return of the cancer, for a defined period of time, such as about 1 year, about 2 years, about 3 years, about 4 years, about 5 years, about 10 years, etc., from initiation of treatment or from initial diagnosis. In one aspect of the invention, DFS is analyzed according to the intent-to-treat principle, i.e., patients are evaluated on the basis of their assigned therapy. The events used in the analysis of DFS can include local, regional and distant recurrence of cancer, occurrence of secondary cancer, and death from any cause in patients without a prior event (e.g., breast cancer recurrence or second primary cancer).

“Overall survival” refers to the patient remaining alive for a defined period of time, such as about 1 year, about 2 years, about 3 years, about 4 years, about 5 years, about 10 years, etc., from initiation of treatment or from initial diagnosis. In the studies underlying the invention the event used for survival analysis was death from any cause.

By “extending survival” is meant increasing DFS and/or OS in a treated patient relative to an untreated patient, or relative to a control treatment protocol. Survival is monitored for at least about six months, or at least about 1 year, or at least about 2 years, or at least about 3 years, or at least about 4 years, or at least about 5 years, or at least about 10 years, etc., following the initiation of treatment or following the initial diagnosis.

By “monotherapy” it is meant a therapeutic regimen that includes only a single therapeutic agent for the treatment of the cancer or tumor during the course of the treatment period.

By “maintenance therapy” or “adjuvant therapy” it is meant a therapeutic regimen that is given to reduce the likelihood of disease recurrence or progression. Maintenance or adjuvant therapy can be provided for any length of time, including extended time periods up to the life-span of the subject. Maintenance or adjuvant therapy can be provided after initial therapy or in conjunction with initial or additional therapies. Dosages used for maintenance therapy or adjuvant therapy can vary and can include diminished dosages as compared to dosages used for other types of therapy.

“Cardiac toxicity” refers to any toxic side effect that affects the heart and that results from administration of a drug or drug combination. Cardiac toxicity can be evaluated based on any one or more of: incidence of symptomatic left ventricular systolic dysfunction (LVSD) or congestive heart failure (CHF), or decrease in left ventricular ejection fraction (LVEF).

The phrases “parenteral administration” and “administered parenterally” are art-recognized terms and include modes of administration other than enteral and topical administration, such as injections, and include, without limitation, intratumoral, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The phrases “systemic administration,” “administered systemically,” “peripheral administration” and “administered peripherally” as used herein mean the administration of a compound, agent or other material other than directly into a specific tissue, organ, or region of the subject being treated (e.g., tumor site), such that it enters the animal's system and, thus, is subject to metabolism and other like processes, for example, subcutaneous administration.

The terms “treat”, “treating”, and “treatment” refer to both therapeutic treatment and prophylactic or preventative measures, wherein the object is to prevent or slow down (lessen) an undesired physiological change or disorder, such as the growth, development or spread of a hyperproliferative condition, such as cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

A “subject”, as used therein, can be a human or non-human animal. Non-human animals include, for example, livestock and pets, such as ovine, bovine, porcine, canine, feline and murine mammals, as well as reptiles, birds and fish. Preferably, the subject is human.

The language “effective amount” or “therapeutically effective amount” refers to a sufficient amount of the composition used in the practice of the invention that is effective to provide effective treatment in a subject, depending on the compound being used. That result can be reduction and/or alleviation of the signs, symptoms, or causes of a disease or disorder, or any other desired alteration of a biological system. An appropriate therapeutic amount in any individual case may be determined by one of ordinary skill in the art using routine experimentation.

A “prophylactic” or “preventive” treatment is a treatment administered to a subject who does not exhibit signs of a disease or disorder, or exhibits only early signs of the disease or disorder, for the purpose of decreasing the risk of developing pathology associated with the disease or disorder.

A “therapeutic” treatment is a treatment administered to a subject who exhibits signs of pathology of a disease or disorder for the purpose of diminishing or eliminating those signs.

“Pharmaceutically acceptable carrier” refers herein to a composition suitable for delivering an active pharmaceutical ingredient, such as the composition of the present invention, to a subject without excessive toxicity or other complications while maintaining the biological activity of the active pharmaceutical ingredient. Protein-stabilizing excipients, such as mannitol, sucrose, polysorbate-80 and phosphate buffers, are typically found in such carriers, although the carriers should not be construed as being limited only to these compounds.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

Embodiments described herein relate to the use of an in situ vaccine that includes plant virus or plant virus-like particles as a neoadjuvant for the treatment of cancer, such as inflammatory breast cancer or inflammatory mammary cancer, in a subject in need thereof. We found that an in situ vaccine that includes plant virus or plant virus-like particles, such as cowpea mosaic virus or virus-like particles, when administered as a neoadjuvant directly or locally to cancer or tumor, such as inflammatory mammary cancer, that is inoperable or unresectable can reduce the cancer or tumor burden in a subject such that the cancer or tumor can be removed or reduced by surgical resection and/or further reduced by radiotherapy. Advantageously, an in situ vaccine administered as a neoadjuvant without the administration or use of a chemotherapeutic can decrease or reduce cancer or tumor burden without adverse events, such as cardiotoxicity, and provide an improved quality of life to cancer patients.

Accordingly, a method of treating cancer in a subject in need thereof can include administering to the subject a neoadjuvant therapy that includes an in situ vaccine and optionally an immune check point therapy prior to surgical resection and/or radiotherapy of the cancer. The in situ vaccine can include at least one of cowpea mosaic virus or cowpea mosaic virus-like particles. The cancer can be treated by surgical resection and/or radiotherapy following administration of the neoadjuvant therapy. Optionally, an adjuvant therapy can be administered to the subject after surgical resection and/or radiotherapy of the cancer.

The in situ vaccination approach does not rely on the plant virus or virus-like particles as a vehicle for drug or antigen delivery, but rather on their inherent immunogenicity. In some embodiments, the in situ administration of the plant virus or plant virus-like particle can be proximal to, or directly adjacent, a tumor site in the subject or directly to the tumor site (e.g., via intratumoral injection) to provide a high local concentration of the plant virus or plant virus-like particle.

### Plant Virus and Plant Virus-Like Particles

The plant virus or plant virus-like particle (VLP) can be nonreplicating and noninfectious in the subject to avoid infection of the subject and can be regarded as safe from a human health and agricultural perspective. In planta production prevents endotoxin contamination that may be a byproduct of other VLP systems derived from E. coli. The VLPs are scalable, stable over a range of temperatures (4-60° C.) and solvent: buffer mixtures.

In some embodiments, plant virus particles or plant virus-like particles in which the viral nucleic acid is not present are administered in situ to cancer of the subject. Virus-like particles lacking their nucleic acid are non-replicating and non-infectious regardless of the subject into which they are introduced.

In other embodiments, the plant virus particles include a nucleic acid within the virus particle. If present, the nucleic acid will typically be the nucleic acid encoding the virus. However, in some embodiments the viral nucleic acid may have been replaced with exogenous nucleic acid. In some embodiments, the nucleic acid is RNA, while in other embodiments the nucleic acid is DNA. A virus particle including nucleic acid will still be nonreplicating and noninfectious when it is introduced into a subject which it cannot infect. For example, plant virus particles will typically be nonreplicating and noninfectious when introduced into an animal subject.

In some embodiments, the plant virus is a plant picornavirus. A plant picornavirus is a virus belonging to the family Secoaviridae, which together with mammalian picornaviruses belong to the order of the Picornavirales. Plant picornaviruses are relatively small, non-enveloped, positive-stranded RNA viruses with an icosahedral capsid. Plant picornaviruses have a number of additional properties that distinguish them from other picornaviruses, and are categorized as a subfamily of Secoviridae. In some embodiments, the virus particles are selected from the Comovirinae virus subfamily. Exemplary Comovirinae subfamily viruses for use in a method described herein can include Cowpea mosaic virus (CPMV), Broad bean wilt virus 1, and Tobacco ringspot virus. In certain embodiments, the plant virus or plant virus-like particles are from the genus Comovirus. A preferred example of a Comovirus is the cowpea mosaic virus or cowpea mosaic virus-like particles. The plant virus-like particle can be an empty cowpea mosaic virus-like particle (eCPMV).

In some embodiments, the plant virus or plant virus-like particle is a rod-shaped plant virus. A rod-shaped plant virus is a virus that primarily infects plants, is non-enveloped, and is shaped as a rigid helical rod with a helical symmetry. Rod shaped viruses also include a central canal. Rod-shaped plant virus particles are distinguished from filamentous plant virus particles as a result of being inflexible, shorter, and thicker in diameter. For example, Virgaviridae have a length of about 200 to about 400 nm, and a diameter of about 15-25 nm. Virgaviridae have other characteristics, such as having a single-stranded RNA positive sense genome with a 3′-tRNA like structure and no polyA tail, and coat proteins of 19-24 kilodaltons.

In some embodiments, the rod-shaped plant virus or virus-like particle belongs to a specific virus family, genus, or species. For example, in some embodiments, the rod-shaped plant virus belongs to the Virgaviridae family. The Virgaviridae family includes the genus Furovirus, Hordevirus, Pecluvirus, Pomovirus, Tobamovirus, and Tobravirus. In some embodiments, the rod-shaped plant virus belongs to the genus Tobamovirus. In further embodiments, the rod-shaped plant virus belongs to the tobacco mosaic virus (TMV) species. The tobacco mosaic virus has a capsid made from 2130 molecules of coat protein and one molecule of genomic single strand RNA 6400 bases long. The coat protein self-assembles into the rod like helical structure (16.3 proteins per helix turn) around the RNA which forms a hairpin loop structure. The protein monomer consists of 158 amino acids which are assembled into four main alpha-helices, which are joined by a prominent loop proximal to the axis of the virion. Virions are ˜300 nm in length and ˜18 nm in diameter. Negatively stained electron microphotographs show a distinct inner channel of ˜4 nm.

In other embodiments, the plant virus or plant virus-like particle is an Alphaflexiviridae virus or virus-like particle. The genera comprising the Alphaflexiviridae family include Allexivirus, Botrexvirus, Lolavirus, Mandarivirus, Potexvirus, and Sclerodarnavirus. In further embodiments, the plant virus particle of the vaccine composition is a Potexvirus particle. Examples of Potexvirus include Allium virus X, Alstroemeria virus X, Alternanthera mosaic virus, Asparagus virus 3, Bamboo mosaic virus, Cactus virus X, Cassava common mosaic virus, Cassava virus X, Clover yellow mosaic virus, Commelina virus X, Cymbidium mosaic virus, Daphne virus X, Foxtail mosaic virus, Hosta virus X, Hydrangea ringspot virus, Lagenaria mild mosaic virus, Lettuce virus X, Lily virus X, Malva mosaic virus, Mint virus X, Narcissus mosaic virus, Nerine virus X, Opuntia virus X, Papaya mosaic virus, Pepino mosaic virus, Phaius virus X, Plantago asiatica mosaic virus, Plantago severe mottle virus, Plantain virus X, Potato aucuba mosaic virus, Potato virus X, Schlumbergera virus X, Strawberry mild yellow edge virus, Tamus red mosaic virus, Tulip virus X, White clover mosaic virus, and Zygocactus virus X. In some embodiments, the plant virus like particle is a Potato virus X virus-like particle.

The plant virus or plant virus-like particles can be obtained according to various methods known to those skilled in the art. In embodiments where plant virus particles are used, the plant virus particles can be obtained from the extract of a plant infected by the plant virus. For example, cowpea mosaic virus can be grown in black eyed pea plants, which can be infected within 10 days of sowing seeds. Plants can be infected by, for example, coating the leaves with a liquid containing the virus, and then rubbing the leaves, preferably in the presence of an abrasive powder which wounds the leaf surface to allow penetration of the leaf and infection of the plant. Within a week or two after infection, leaves are harvested and viral nanoparticles are extracted. In the case of cowpea mosaic virus, 100 mg of virus can be obtained from as few as 50 plants. Procedures for obtaining plant picornavirus particles using extraction of an infected plant are known to those skilled in the art. See Wellink J., Meth Mol Biol, 8, 205-209 (1998). Procedures are also available for obtaining virus-like particles. Saunders et al., Virology, 393 (2): 329-37 (2009). The disclosures of both of these references are incorporated herein by reference.

### Immune Checkpoint Therapy

In some embodiments, the neoadjuvant therapy can include the administration of a plant virus or plant virus-like particles and an immune checkpoint therapy or therapeutic prior to surgical resection of the cancer and/or adjuvant therapy with, for example, a chemotherapeutic. The combination of an immune checkpoint therapy and a plant virus or plant virus-like particle can allow for a lower systemic dose of immune checkpoint therapy, mitigating the impact of adverse events, typical of immune checkpoint therapies.

Immune checkpoint therapy for cancer encompasses strategies that target immunity regulatory pathways in order to enhance immunity activity against tumor cells. It has been shown that treatment with a plant virus and immune checkpoint-targeting antibodies, such as anti-PD-1 antibodies, anti-PD-L1 antibodies, or agonistic OX40-specific antibodies increases tumor infiltration by antitumor neutrophils/macrophages, natural killer cells, and CD4+ and CD8+ effector T cells secreting greater amounts of interferon gamma, while depleting myeloid-derived suppressive cells and regulatory T cells. Thus, in some embodiments, immune checkpoint therapy can include the administration to a subject of one or more immune checkpoint modulating agents to eradicate suppressive regulatory T cells and/or initiate an effector immune response with the in situ administration of the plant virus or virus-like particle.

An immune checkpoint therapeutic for use in a method described herein can include an agent that either inhibits negative regulators of the immune system response against cancer cells, such as programmed cell death protein 1 (PD-1) or PD-L1, or agents that act as agonists for positive regulators, such as OX40 (CD134). In particular embodiments, the immune checkpoint modulating agents can include an immune checkpoint-targeting antibody such as anti-PD-1, anti-PD-L1, or agonistic OX40-specific monoclonal antibodies.

The programmed death 1 (PD-1) immune checkpoints are negative regulators of T-cell immune function and inhibition of PD-1/PD-L1, results in increased activation of the immune system. In some embodiments, an immune checkpoint therapeutic administered to a subject can include a PD-1/PD-L1 inhibitor. In certain embodiments, the PD-1 inhibitor in an anti-PD-1 antibody. Anti-PD-1 antibodies for use in a method described herein can include monoclonal antibodies capable of inhibiting the engagement/interaction of PD-1 with PD-L1 ligand. Thus, in some embodiments, a PD-1 inhibitory agent can include an antibody that targets PD-L1.

Exemplary PD-1 inhibitory monoclonal antibodies for use in a combination therapy described herein include, but are not limited to, Pembrolizumab, Nivolumab, and Cemiplimab, and MEDI0608. Exemplary PD-L1 inhibitory monoclonal antibodies include, but are not limited to, Atezolizumab, Avelumab, Vonlerolizumab, and Durvalumab.

Additional immune checkpoint therapeutics that target negative regulators of the immune system response against cancer cells can include agents capable of inhibiting or blocking engagement/interaction with Cytotoxic T-lymphocyte-associated antigen 4 (CTLA-4), lymphocyte activation gene 3 (LAG-3, CD223), T cell immunoglobulin-3 (TIM-3), T cell immunoglobulin and ITIM domain (TIGIT), V-domain Ig suppressor of T cell activation (VISTA), and B7 homolog 3 (B7/H3).

For example, an immune checkpoint therapeutics targeting CTLA-4 can include the anti-CTLA-4 antibodies Tremelimunab, BMS-986249, and Ipilimumab, which is approved for the treatment of advanced or unresectable melanoma. Agents targeting LAG-3 can include the IMP321 fusion protein and monoclonal antibodies targeting LAG-3, such as Relatlimab or LAG525. An agent targeting TIM-3 can include the anti-TIM-3 monoclonal antibody MBG453. An agent targeting TIGIT can include the anti-TIGIT monoclonal antibody OMP-31M32. Agents targeting VISTA, also known as programmed death-1 homolog (PD-1H), can include human monoclonal antibody JNJ-61610588 and CA-170, an oral inhibitor of both PD-L1/PD-L2 and VISTA. Agents targeting B7-H3, also known as CD276, can include Enoblituzumab (MGA271) which is an engineered Fc humanized IgG1 monoclonal antibody against B7-H3, the humanized DART protein MGD009, and 8H9 which is an antibody against B7-H3 labeled with radioactive iodine (I-131) which, after internalization, promotes cancer cell death.

In some embodiments, the immune checkpoint therapeutic can include a positive regulator of the immune system response against cancer cells. In particular embodiments, immune checkpoint therapeutics that act as positive regulators of the immune system response against cancer cells can include OX40 agonistic agents. In an exemplary embodiment, an OX40 agonistic agent can include a monoclonal antibody capable of promoting the engagement/interaction of OX40 with OX40L ligand to promote the NF-κB signaling pathway and T cell clonal expansion and activation. OX40 agnostic agents for use in a method described herein can include, but are not limited to, MEDI6368 fusion protein, MEDI0562, MEDI6469, BMS986178, Pf-04518600 (PF-8600), GSK3174998 and MOXR0916.

Additional immune checkpoint therapeutics for use in a method described herein can include agonistic agents targeting positive regulators of the immune system response against cancer cells such as, but not limited to, Inducible co-stimulator (ICOS), Glucocorticoid-induced TNF receptor family-related protein (GITR), 4-1BB, CD27/CD70 pathway, and CD40. GITR agonists can include TRX-518, an aglycosylated human mAb, BMS-986156, AMG 228, MEDI1873, MK-4166, INCAGN01876, and GWN323. ICOS agonists can include JTX-2011, GSK3359609, and MEDI-570. 4-1BB (CD137) agonists can include Utomilumab (PF-05082566) and Urelumab. Agonists of the CD27/CD70 pathway can include ARGX-110, BMS-936561 (MDX-1203), and Varlilumab. CD40 agonists can include CP-870893, APX005M, ADC-1013, lucatumumab, Chi Lob 7/4, dacetuzumab, SEA-CD40, and RO7009789 monoclonal antibodies.

Additional exemplary immune checkpoint therapeutics can include, but are not limited to, agents capable of inhibiting or blocking engagement/interaction with adenosine A2a receptor (A2aR), CD73, B and T cell lymphocyte attenuator (BTLA, CD272), or non-T cell-associated inhibitory molecules such as transforming growth factor β(TGF-β), Killer immunoglobulin-like receptors (KIRs, CD158), Phosphoinositide 3-kinase gamma (PI3Kγ), and CD47 (integrin-associated protein).

Further immune checkpoint therapeutics can include molecules targeting tumor microenvironment components like Indoleamine 2,3-dioxygenase (IDO), Toll-like receptors (TLRs), IL2R, as well as arginase inhibitors such as CB-1158 or oncolytic peptides, such as LTX-315. For example agents targeting (IDO) can include BMS-986205, and Indoximod, and the oral agent epacadostat. Agents targeting TLRs for use as an immune checkpoint therapeutic in a method described herein can include MEDI9197, PG545 (pixatimod, pINN), and Polyinosinic-polycytidylic acid polylysine carboxymethylcellulose (poly-ICLC). For example, an IL-2R inhibitory agent can include NKTR-214 (bempeg) and an IL-10 inhibitory agent can include AM0010 (pegilodecakin).

In some embodiments, selection of particular immune checkpoint therapy for use in a method described herein can be achieved by determining the expression of immune checkpoint molecules generated using a plant virus (e.g., CPMV) on CD4+ effector T cells of a subject, where the increased expression of immune checkpoint molecules can predict the potency of specific immune checkpoint modulating agents.

### Cancer Treatment by Neoadjuvant Therapy

In a method of neoadjuvant therapy, the in situ vaccine and optionally an immune checkpoint therapeutic is administered prior to an individual undergoing treatment by surgery or radiation to reduce the amount of cancer or tumor in the subject.

Any cancer or tumor can be treated by this method of neoadjuvant therapy, including both pediatric and adult tumors. The cancers treated by a method described herein can include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, glioblastoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytoma and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, fallopian tube cancer, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In some embodiments, the cancer can be a locally advanced, metastatic, and/or inflammatory cancer.

In other embodiments, the cancer can be inflammatory breast or mammary cancer, such as stage III or stage IV inflammatory breast or mammarycancer. In particular embodiments, the cancer treated in accordance with a method described herein can include a cancer characterized by tumors with low immunogenicity. In certain embodiments, cancers with low immunogenicity treated using a neoadjuvant therapy, such as a combination of a plant virus or plant virus-like particle in situ vaccine and an optional immune checkpoint therapeutic, can include breast cancer with low immunogenicity. In exemplary embodiments, a combination of CPMV or CPMV virus-like particles and an anti-PD-1 antibody or anti-PD-L1 antibody can be used to treat breast cancer cancer characterized by tumors with low immunogenicity, such as PD-L1+ inflammatory breast cancer.

Treatment With Plant Virus or Plant Virus-Like Particles and Optional Immune Checkpoint Therapeutic Followed by Surgical Resection and/or Radiotherapy

The in situ vaccine, which includes the plant virus or plant virus-like particles, and optional immune checkpoint therapy, alone as a monotherapy or in combination, can be administered as a pharmaceutical composition, comprising a mixture, and a pharmaceutically acceptable carrier. The plant virus or virus-like particles and optional an immune checkpoint modulating agent may be present in a pharmaceutical composition in an amount from 0.001 to 99.9 wt %, more preferably from about 0.01 to 99 wt %, and even more preferably from 0.1 to 95 wt %.

The plant virus particles or virus-like particles and optional immune checkpoint therapy, or pharmaceutical compositions comprising these particles or agents, may be administered by any method designed to provide the desired effect. In the methods described herein, any technique for directly administering an in situ vaccine to the tumor may be used. Direct administration does not rely on the blood vasculature to access the tumor. The preparation may be applied on the surface of the tumor, injected into the tumor, instilled in or at the tumor site during surgery, infused into the tumor via a catheter, etc. In a particular embodiment, the plant virus or virus-like particle and the immune checkpoint therapeutic are administered to the subject by intratumoral injection.

The inherent immunogenicity resulting from an in situ vaccination approach described herein appears to be uniquely potent when the plant virus or virus-like particles are inhaled or when administered through intratumoral administration or as IP administration when treating metastatic cancer. For treatment of breast cancer, the plant virus or virus-like particles can be directly injected into a breast tumor or breast cancer of the subject with established breast tumors and this immunostimulatory treatment results in the rejection of those tumors and systemic immunity that prevents growth of distal tumors. The plant virus or virus-like particles described herein (e.g., CPMV) alone are able to stimulate systemic anti-tumor immunity. The in situ administration of a plant virus or virus-like particles and systemic and/or local administration of an immune checkpoint therapeutic can render the tumor microenvironment inhospitable to tumor cell seeding or continued growth and provide a synergistic effect on the antitumor response at a tumor site of the subject.

An immune checkpoint therapeutic, such as an immune checkpoint inhibitor, may be administered by any appropriate means known in the art for the particular inhibitor. These include systemic and local administration, such as intravenous, oral, intraperitoneal, sublingual, intrathecal, intracavitary, intramuscularly, intratumorally, and subcutaneously. Optionally, the immune checkpoint therapeutic may be administered in combination with an in situ vaccine directly to the cancer or tumor.

When formulated as separate compositions, “combination therapy” is intended to embrace administration of these therapeutic agents in a sequential manner, that is, wherein each therapeutic agent is administered at a different time, as well as administration of these therapeutic agents, in a substantially simultaneous manner. For example, administration of the plant virus or plant virus-like particles can be carried out in a substantially simultaneous manner as the one or more immune checkpoint therapeutic administration. Sequential or substantially simultaneous administration of each therapeutic agent can be effected by any appropriate route including, but not limited to, intratumoral routes, intraperitoneal routes, oral routes, intravenous routes, intramuscular routes, and direct absorption through mucous membrane tissues. A preferred method for administering the plant virus or virus-like particle and one or more immune checkpoint therapeutics to a subject having cancer is by intratumoral injection. However, the therapeutic agents can be administered by the same route or by different routes. For example, plant virus or plant virus-like particles of the combination selected may be administered by intratumoral injection while the immune checkpoint therapeutic(s) of the combination may be administered orally or intravenously. Alternatively, for example, all therapeutic agents may be administered by intratumorally injection. The sequence in which the therapeutic agents are administered is not narrowly critical.

The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Suitable pharmaceutically acceptable carriers may contain inert ingredients which do not unduly inhibit the biological activity of the compounds. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

A pharmaceutically acceptable carrier for a pharmaceutical composition can also include delivery systems known to the art for entraining or encapsulating drugs, such as anticancer drugs. In some embodiments, the disclosed compounds can be employed with such delivery systems including, for example, liposomes, nanoparticles, nanospheres, nanodiscs, dendrimers, and the like. See, for example Farokhzad, O. C., Jon, S., Khademhosseini, A., Tran, T. N., Lavan, D. A., and Langer, R. (2004). “Nanoparticle-aptamer bioconjugates: a new approach for targeting prostate cancer cells.” Cancer Res., 64, 7668-72; Dass, C. R. (2002). “Vehicles for oligonucleotide delivery to tumours.” J. Pharm. Pharmacol., 54, 3-27; Lysik, M. A., and Wu-Pong, S. (2003). “Innovations in oligonucleotide drug delivery.” J. Pharm. Sci., 92, 1559-73; Shoji, Y., and Nakashima, H. (2004). “Current status of delivery systems to improve target efficacy of oligonucleotides.” Curr. Pharm. Des., 10, 785-96; Allen, T. M., and Cullis, P. R. (2004). “Drug delivery systems: entering the mainstream.” Science, 303, 1818-22. The entire teachings of each reference cited in this paragraph are incorporated herein by reference.

The formulations may be conveniently presented in unit dosage form and may be prepared by any of the methods well known in the art of pharmacy. Preferably, such methods include the step of bringing the virus particles into association with a pharmaceutically acceptable carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The methods of the invention include administering to a subject, preferably a mammal, and more preferably a human, the composition of the invention in an amount effective to produce the desired effect.

One skilled in the art can readily determine an effective amount of plant virus or plant virus-like particles and optional immune checkpoint therapeutic to be administered to a given subject, by taking into account factors such as the size and weight of the subject; the extent of disease penetration; the age, health and sex of the subject; the route of administration; and whether the administration is local or systemic. Those skilled in the art may derive appropriate dosages and schedules of administration to suit the specific circumstances and needs of the subject. For example, suitable doses of the virus particles to be administered can be estimated from the volume of cancer cells to be killed or volume of tumor to which the virus particles are being administered.

Useful dosages of the active agents can be determined by comparing their in vitro activity and the in vivo activity in animal models. Methods for extrapolation of effective dosages in mice, and other animals, to humans are known in the art. An amount adequate to accomplish therapeutic or prophylactic treatment is defined as a therapeutically- or prophylactically-effective dose. In both prophylactic and therapeutic regimes, agents are usually administered in several dosages until an effect has been achieved. Effective doses of the virus particles vary depending upon many different factors, including means of administration, target site, physiological state of the patient, whether the patient is human or an animal, characteristics of the subject, such as general health, age, sex, body weight and tolerance to drugs as well as the degree, severity and type of cancer, other medications administered, and whether treatment is prophylactic or therapeutic. The skilled artisan will be able to determine appropriate dosages depending on these and other factors using standard clinical techniques.

In some embodiments, the method includes administering in situ a plant virus or plant virus-like particle to the subject in combination with one or more immune checkpoint therapeutics. The immune checkpoint therapeutics can target the same immune checkpoint or can target two or more immune checkpoints. In an exemplary embodiment, a method of treating cancer in a subject can include administering in situ to the subject a therapeutically effective amount of a cowpea mosaic virus or cowpea mosaic virus-like particle in combination with a PD-1 inhibitor, PD-L1 inhibitor, and/or an OX40 agonist.

The therapeutically effective amounts of neoadjuvant therapy can include an amount(s) effective to increase tumor infiltration by antitumor neutrophils/macrophages, natural killer cells, an/or CD4+ and CD8+ effector T cells and inhibit immunosuppressive cells in the tumor microenvironment. In some embodiments, a therapeutically effective amount of a neoadjuvant, which includes an in situ vaccine comprising the plant virus or plant virus-like particles and an optional immune checkpoint therapy, can include an amount effective to generate systemic tumor-specific T cells targeting the subject's tumor cells. In an exemplary embodiment, a therapeutically effective amount of a neoadjuvant can be effective to reduce cancer or tumor burden in the subject such that the cancer or tumor is operable by surgical resection.

In other embodiments, the neoadjuvant therapy can be administered at an amount effective to decrease Treg/CD8+ ratio and/or increase CD8+GZMB+ T cell level in blood of the subject.

In some embodiments, the cancer can be inflammatory breast or mammary cancer, which is inoperable prior to administration of the neoadjuvant therapy and optional immune check point therapy, and the neoadjuvant therapy and optional immune checkpoint therapy is administered at amount effective for the inflammatory breast or mammary cancer to be operable by surgical resection.

The methods described herein contemplate single as well as multiple administrations, given either simultaneously or over an extended period of time. A pharmaceutically acceptable composition containing the neoadjuvant can be administered at regular intervals, depending on the nature and extent of the cancer's effects, and on an ongoing basis. Administration at a “regular interval,” as used herein, indicates that the therapeutically effective amount is administered periodically (as distinguished from a one-time dose). In one embodiment, the pharmaceutically acceptable composition including the plant virus, virus-like particle, one or more immune checkpoint modulating agents, and optionally an additional cancer therapeutic is administered periodically, e.g., at a regular interval (e.g., bimonthly, monthly, biweekly, weekly, twice weekly, daily, twice a day or three times or more often a day). In an exemplary embodiment, the pharmaceutically acceptable composition is administered to the subject weekly.

The administration interval for a single individual can be fixed, or can be varied over time, depending on the needs of the individual. For example, in times of physical illness or stress, or if disease symptoms worsen, the interval between doses can be decreased.

For example, the administration of a plant virus or virus like particle and/or optional immune checkpoint therapy can take place at least once on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least once on week 1, 2, 3, 4, 5, 6, 7, 8, 9, 10, 11, 12, 13, 14, 15, 16, 17, 18, 19 or 20, or any combination thereof, using single or divided doses of every 60, 48, 36, 24, 12, 8, 6, 4, or 2 hours, or any combination thereof. Administration can take place at any time of day, for example, in the morning, the afternoon or evening. For instance, the administration can take place in the morning, e.g., between 6:00 a.m. and 12:00 noon; in the afternoon, e.g., after noon and before 6:00 p.m.; or in the evening, e.g., between 6:01 p.m. and midnight.

Typically, wherein the neoadjuvant therapy comprises an in situ vaccine and an immune checkpoint therapeutic, the two agents will be administered within days of each other. For example, the in situ vaccine can administered followed by administration of immune checkpoint therapeutic at 30, 28, 21, 14, 10, 9, 8, 7, 6, 5, 4, 3, 2, or 1 day(s) after administration of the immune checkpoint inhibitor. Alternatively, it may be advantageous to administer the immune check point therapeutic prior to administration of the in situ vaccine, wherein the in situ vaccine is then administered to the individual within several days or weeks (e.g., at 30, 28, 21, 14, 10, 9, 8, 7, 6, 5, 4, 3, 2, or 1 day(s)) after administration of the immune check point therapy. Priming of a cytotoxic T lymphocyte response by the in situ vaccine may take from about 5 to about 14 days. Administration of the immune checkpoint therapeutic may beneficially be commenced before, during, or after such priming period. For example, in one aspect, the immune checkpoint therapeutic is administered 14 days after administration of the in situ vaccine, and after about 1 week to about 3 weeks following administration of the immune checkpoint inhibitor, the individual is then treated to reduce tumor burden (e.g., by surgery or radiation therapy).

Typically, wherein the neoadjuvant therapy comprises administration of an in situ vaccine, about 1 week to about 3 weeks later after receiving the in situ vaccine, the individual is then treated to reduce tumor burden (e.g., by surgery or radiation therapy). Optionally, following reduction of tumor burden, the individual may receive maintenance therapy with the in situ vaccine and/or immune check point therapeutic which comprised periodic (e.g., about every 1 week to 3 weeks) administration of a therapeutically effective amount of an immune checkpoint therapeutic, and/or may be administered in combination with the in situ vaccine should the tumor recur.

Optionally, the individual may receive maintenance therapy with the in situ vaccine and/or immune check point therapeutic which comprised periodic (e.g., about every 1 week to 3 weeks) administration of a therapeutically effective amount of an immune checkpoint therapeutic, and/or may be administered in combination with the in situ vaccine should the tumor recur.

In some embodiments, surgery and/or radiotherapy can be performed no later than about 1, about 2, about 3, about 4, about 5, about 6, about 7, or about 8 weeks following the last administration of neoadjuvant therapy. By way of example, for breast cancer, after completion of neoadjuvant therapy, the patient can undergo a mastectomy or a wide local excision procedure such as segmental/partial mastectomy)

### Adjuvant Therapy

In addition to neoadjuvant therapy comprising administering the in situ vaccine and the optional one or more immune checkpoint therapeutics followed by surgical removal of the cancer or tumor or reduction of the cancer or tumor by radiotherapy, treatment of the subject may comprise administration of one or more adjuvant therapy or maintenance therapy, such as chemotherapy, biological therapy, immunotherapy, ultrasound therapy, or radiotherapy following surgical resection or reduction by radiotherapy. These modalities may be current standard of care for treatment of certain human tumors. The neoadjuvant therapy may be administered before, during, or after the standard of care for treating the tumor. For example, in situ vaccine and optional immune checkpoint inhibitor combination comprising neoadjuvant therapy may be administered after failure of the standard of care. When a combination of immunotherapeutic agents is specified, each agent may be administered separately in time as two separate agents within a single combination regimen. Alternatively, the two (or more) agents may be administered in admixture.

In some embodiments, the method can further include the step of administering a therapeutically effective amount of an adjuvant anticancer therapeutic agent to the subject. The anticancer therapeutic agents can be in the form of biologically active ligands, small molecules, peptides, polypeptides, proteins, DNA fragments, DNA plasmids, interfering RNA molecules, such as siRNAs, oligonucleotides, and DNA encoding for shRNA. In some embodiments, cytotoxic compounds are included in an anticancer agent described herein. Cytotoxic compounds include small-molecule drugs such as doxorubicin, mitoxantrone, methotrexate, and pyrimidine and purine analogs, referred to herein as antitumor agents.

The anticancer therapeutic agent can include an anticancer or an antiproliferative agent that exerts an antineoplastic, chemotherapeutic, antiviral, antimitotic, antitumorgenic, and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agent agents available in commercial use, in clinical evaluation and in pre-clinical development. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids.

The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

Examples of anticancer therapeutic agents that can be administered after neoadjuvant administration and surgical resection or reduction of the cancer as an adjuvant in an adjuvant therapy include Taxol, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon-α-2a; interferon alfa-2b; interferon alfa-n1; interferon alfa-n3; interferon beta-Ia; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; temozolomide, teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anticancer therapeutic agents include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; silicon phthalocyanine (PC4) sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosamOinoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

Other anticancer agents can include the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-223651), SAH-49960 (Lilly/Novartis), SDZ-268970 (Lilly/Novartis), AM-97 (Armad/Kyowa Hakko), AM-132 (Arnad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin Al (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-IAABE (Cytoskeleton/Mt. Sinai School of Medicine), A-204197 (Abbott), T-607 (Tularik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NC1), Resverastatin phosphate sodium, BPR-OY-007 (National Health Research Institutes), and SSR-250411 (Sanofi).

Still other anticancer therapeutic agents include alkylating agents, such as nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.), antimetabolites, such as folic acid analog (e.g., methotrexate), or pyrimidine analogs (e.g., fluorouracil, floxouridine, Cytarabine), purine analogs (e.g., mercaptopurine, thioguanine, pentostatin, vinca alkaloids (e.g., vinblastin, vincristine), epipodophyllotoxins (e.g., etoposide, teniposide), platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, amino glutethimide).

In particular embodiments, anticancer agents include angiogenesis inhibitors such as angiostatin K1-3, DL-α-difluoromethyl-ornithine, endostatin, fumagillin, genistein, minocycline, staurosporine, and (±)-thalidomide; DNA intercalating or cross-linking agents such as bleomycin, carboplatin, carmustine, chlorambucil, cyclophosphamide, cisplatin, melphalan, mitoxantrone, and oxaliplatin; DNA synthesis inhibitors such as methotrexate, 3-Amino-1,2,4-benzotriazine 1,4-dioxide, aminopterin, cytosine β-D-arabinofuranoside, 5-Fluoro-5′-deoxyuridine, 5-Fluorouracil, gaciclovir, hydroxyurea, and mitomycin C; DNA-RNA transcription regulators such as actinomycin D, daunorubicin, doxorubicin, homoharringtonine, and idarubicin; enzyme inhibitors such as S(+)-camptothecin, curcumin, (−)-deguelin, 5,6-dichlorobenz-imidazole 1-β-D-ribofuranoside, etoposine, formestane, fostriecin, hispidin, cyclocreatine, mevinolin, trichostatin A, tyrophostin AG 34, and tyrophostin AG 879, Gene Regulating agents such as 5-aza-2′-deoxycitidine, 5-azacytidine, cholecalciferol, 4-hydroxytamoxifen, melatonin, mifepristone, raloxifene, all trans-retinal, all trans retinoic acid, 9-cis-retinoic acid, retinol, tamoxifen, and troglitazone; Microtubule Inhibitors such as colchicine, dolostatin 15, nocodazole, paclitaxel, podophyllotoxin, rhizoxin, vinblastine, vincristine, vindesine, and vinorelbine; and various other antitumor agents such as 17-(allylamino)-17-demethoxygeldanamycin, 4-Amino-1,8-naphthalimide, apigenin, brefeldin A, cimetidine, dichloromethylene-diphosphonic acid, leuprolide, luteinizing-hormone-releasing hormone, pifithrin, rapamycin, thapsigargin, and bikunin, and derivatives (as defined for imaging agents) thereof.

In some embodiments, the adjuvant therapy can further include ablating the cancer. Ablating the cancer can be accomplished using a method selected from the group consisting of cryoablation, thermal ablation, radiotherapy, chemotherapy, radiofrequency ablation, electroporation, alcohol ablation, high intensity focused ultrasound, photodynamic therapy, administration of monoclonal antibodies, immunotherapy, and administration of immunotoxins.

In some embodiments, the step ablating the cancer includes immunotherapy of the cancer. Cancer immunotherapy is based on therapeutic interventions that aim to utilize the immune system to combat malignant diseases. It can be divided into unspecific approaches and specific approaches. Unspecific cancer immunotherapy aims at activating parts of the immune system generally, such as treatment with specific cytokines known to be effective in cancer immunotherapy (e.g., IL-2, interferon's, cytokine inducers).

In contrast, specific cancer immunotherapy is based on certain antigens that are preferentially or solely expressed on cancer cells or predominantly expressed by other cells in the context of malignant disease (usually in vicinity of the tumor site). Specific cancer immunotherapy can be grouped into passive and active approaches.

In passive specific cancer immunotherapy substances with specificity for certain structures related to cancer that are derived from components of the immune system are administered to the patient. The most prominent and successful approaches are treatments with humanized or mouse/human chimeric monoclonal antibodies against defined cancer associated structures (such as Trastuzumab, Rituximab, Cetuximab, Bevacizumab, Alemtuzumab). The pharmacologically active substance exerts is activity as long as a sufficient concentration is present in the body of the patient, therefore administrations have to be repeated based on pharmacokinetic and pharmacodynamic considerations.

On the other hand, active specific cancer immunotherapy aims at antigen-specific stimulation of the patient's immune system to recognize and destroy cancer cells. Active specific cancer immunotherapy, therefore, in general, is a therapeutic vaccination approach. There are many types of cancer vaccine approaches being pursued, such as vaccination with autologous or allogeneic whole tumor cells (in most cases genetically modified for better immune recognition), tumor cell lysates, whole tumor associated antigens (produced by means of genetic engineering or by chemical synthesis), peptides derived from protein antigens, DNA vaccines encoding for tumor associated antigens, surrogates of tumor antigens such as anti-idiotypic antibodies used as vaccine antigens, and the like. These manifold approaches are usually administered together with appropriate vaccine adjuvants and other immunomodulators in order to elicit a quantitatively and qualitatively sufficient immune response (many novel vaccine adjuvant approaches are being pursued in parallel with the development of cancer vaccines). Another set of cancer vaccine approaches relies on manipulating dendritic cells (DC) as the most important antigen presenting cell of the immune system. For example, loading with tumor antigens or tumor cell lysates, transfection with genes encoding for tumor antigens and in-vivo targeting are suitable immunotherapies that can be used together with the virus or virus-like particles of the invention for cancer treatment.

In some embodiments, the step of ablating the cancer includes administering a therapeutically effective amount of radiotherapy (RT) to the subject. It is contemplated that the treatment of the subject or cancer with radiotherapy following neoadjuvant administration and surgical resection or reduction can result in significantly reduced tumor growth compared to RT or CPMV-immune checkpoint modulating agent treatment alone. Without being bound by theory, it is believed that RT can prime the tumor by debulking the tumor to provide a burst of tumor antigens in the context of immunogenic cell death that fosters specific immune recognition and response to those antigen; in turn, plant virus nanoparticle-mediated immune stimulation can further augment antitumor immunity to protect from outgrowth of metastases and recurrence of the disease.

Radiotherapy uses high-energy rays, such as x-rays and similar rays (e.g., electrons) to treat cancer. Radiotherapy administered to a subject can include both external and internal. External radiotherapy (or external beam radiation) aims high-energy x-rays at the tumor site including in some cases the peri-tumor margin. External radiotherapy typically includes the use of a linear accelerator (e.g., a Varian 2100C linear accelerator). External radiation therapy can include three-dimensional conformal radiation therapy (3D-CRT), image guided radiation therapy (IGRT), intensity modulated radiation therapy (IMRT), helical-tomotherapy, photon beam radiation therapy, proton beam radiation therapy, stereotactic radiosurgery and/or sterotactic body radiation therapy (SBRT).

Internal radiotherapy (brachytherapy) involves having radioactive material placed inside the body and allows a higher dose of radiation in a smaller area than might be possible with external radiation treatment. It uses a radiation source that's usually sealed in an implant. Exemplary implants include pellets, seeds, ribbons, wires, needles, capsules, balloons, or tubes. Implants are placed in your body, very close to or inside the tumor. Internal radiotherapy can include intracavitary or interstitial radiation. During intracavitary radiation, the radioactive source is placed in a body cavity (space), such as the uterus. With interstitial radiation, the implants are placed in or near the tumor, but not in a body cavity.

Examples have been included to more clearly describe particular embodiments of the invention. However, there are a wide variety of other embodiments within the scope of the present invention, which should not be limited to the particular examples provided herein.

### EXAMPLE

In this Example, we evaluated the clinical efficacy of the highly immunogenic eCPMV nanoparticles against canine inflammatory mammary cancer (IMC). CPMV and eCPMV are identical in their protein content, but eCPMV lacks RNA and, therefore, is non-infectious toward plants, offering safety from an agricultural perspective. Our results demonstrated robust clinical efficacy of neoadjuvant in situ eCPMV immunotherapy (eCPMV immunotherapy from here on) leading to tumor reduction in all treated IMC dogs and improved survival of dogs treated with this agent. eCPMV immunotherapy induced a strong neutrophilic tumor infiltration associated with necrosis and supported neutrophils as a driver of tumor cell death.

### Materials and Methods

### Canine Patient Recruitment and Selection Criteria

Diagnosis of IMC was based on the presence of characteristic clinical signs including erythema, edema, warmth, pain, and histopathological confirmation of neoplastic invasion of superficial dermal lymphatic vessels. The characteristics of the 10 IMC patients included in this study are summarized in Table 1. All patients' owners signed an informed consent. For this study, we followed the ARRIVE guidelines 2.0.

eCPMV Immunotherapy Protocol and Follow-Up

A pretreatment incisional biopsy of the target tumor was taken from all patients followed by intratumoral eCPMV injection immediately after obtaining the biopsy sample. Collection of the tumor biopsy is described in online supplemental file 1. Incisional biopsies from two patients with confirmed IMC status referred from a different institution were not available. eCPMV production, dosage, number of the tumor injections, and the tumor injection procedure are described in FIG. 5. In situ eCPMV vaccination was administered once a week (day 0 (D0) and day 7 (D7)). Reduction in tumor volume (Tv) was observed by D14 and a few owners of IMC patients agreed to additional eCPMV immuno-therapy (Table 2). The IMC patients treated in this study presented one (patients P3, P4 and P5) or two individual tumor masses (patients P1 and P2). For P1, only the larger tumor was injected during the first two treatments, and both tumors were injected in the subsequent treatments. For P2, both tumors were always injected.

IMC patients are not eligible for a surgical procedure. The medical therapy was added to eCPMV immunotherapy after the second eCPMV injection. Patient P1 had severe adverse events to toceranib and was taken off medical therapy after 23 days of administration. After diagnosis, all IMC control dogs not treated with eCPMV received the medical therapy until death (Table 1).

When eCPMV-induced tumor reduction allowed surgery, the tumor was resected with collection of a surgical biopsy, and the medical therapy was maintained as adjuvant treatment. Surgical procedures were performed per institutional standard of care protocol. After surgery, follow-up was performed once a month until death or euthanasia. Thoracic radiographs and abdominal ultra-sound were performed every 2 months to search for distant metastases.

### Quality of Life and Tumor Response Evaluation

Each canine patient was closely observed by the attending veterinarian in the clinic for 4 hours after each injection and subsequently by the owner on a daily basis to evaluate the potential adverse events induced by eCPMV vaccination. The quality of life (QOL) of each patient was evaluated at first eCPMV dose and at D7 and D14 using a preestablished survey.

The tumor response to the eCPMV vaccination was evaluated once a week during the treatment period by measuring the Tv with calipers using the formula Tv=0.5×long axis x short axis. The percentage of tumor growth inhibition (% TGI) was estimated by the formula % TGI=100×(final Tv-initial Tv)/initial Tv. All measurements are in cubic centimeters (cm³). Although the Response Evaluation Criteria in Solid Tumors in cancer immunotherapy trials (iRECIST) is not designed to address in situ vaccination in canine patients, the iRECIST criteria are also provided in Table 4.

TABLE 1
Epidemiological and clinicopathological characteristics of eCPMV-treated and control canine IMC patients

| | | | | | | | | | | | | |
|---|---|---|---|---|---|---|---|---|---|---|---|---|
| | | | | | | | Histo. | Histo. | | | | |
| Patient | Age, y. | Weight, kg | TS,* cm | TS,† cm³ | Breed | Type | grade | type | sdLVI | LNI | Therapy | OS, days |

eCPMV-treated IMC patients

| | | | | | | | | | | | | |
|---|---|---|---|---|---|---|---|---|---|---|---|---|
| P1 | 11.0 | 10.3 | 15.1 | 74.8 | Mixed | Primary | III | Special | Yes | Yes | +FCT | 174 |
| | | | | | | | | type | | | | |
| P2 | 13.5 | 25.6 | 15.7 | 45.7 | Mixed | Secondary | III | Simple | Yes | Yes | +FCT | 156 |
| P3 | 10.7 | 8.2 | 19.6 | 104.2 | Poodle | Secondary | III | Simple | Yes | Yes | +FCT | 109 |
| P4 | 10.7 | 17.0 | 17.3 | 52.7 | Kerry Blue | Secondary | III | Simple | Yes | Yes | +FCT | 165 |
| | | | | | Terrier | | | | | | | |
| P5 | 11.9 | 2.7 | 3.9 | 4.3 | Bichon Frise | Secondary | III | Simple | Yes | Yes | +FCT | 67 |

Control IMC patients

| | | | | | | | | | | | | |
|---|---|---|---|---|---|---|---|---|---|---|---|---|
| P6 | 13.0 | 26.2 | 14.0 | 252.0 | Mixed | Secondary | III | DA | Yes | Yes | FCT | 27 |
| P7 | 14.2 | 7.6 | 7.0 | 14.0 | Maltese | Secondary | III | Simple | Yes | Yes | FCT | 40 |
| P8 | 9.7 | 10.3 | 4.0 | 32.0 | Mixed | Secondary | III | Simple | Yes | Yes | FCT | 132 |
| P9 | 13.0 | 9.3 | 5.0 | 30.6 | Miniature | Secondary | III | Simple | Yes | Yes | FCT | 63 |
| | | | | | Schnauzer | | | | | | | |
| P10 | 8.9 | 7.7 | 3.0 | 9.4 | Poodle | Secondary | III | Simple | Yes | Yes | FCT | 73 |

Age at diagnosis in years.
+FCT indicates that eCPMV-treated dogs received FCT therapy starting after second eCPMV injection until surgery or death, and P1 and P5 continued on FCT as adjuvant therapy until death.
*TS refers to the largest diameter of the target tumor in cm.
†TS refers to tumor volume in cm3; type, refers to primary and secondary IMC.
DA, ductal associated; eCPMV, empty cowpea mosaic virus; FCT, firocoxib + cyclophosphamide + toceranib; Histo, histologic; IMC, inflammatory mammary cancer; LNI, regional lymph node involvement; OS, overall survival time; sdL VI, superficial dermal lymphovascular invasion; TS, tumor size.

TABLE 2
Tv changes in eCPMV-treated and control dogs

| | | eCPMV | | Tv, | % | |
|---|---|---|---|---|---|---|
| Patient | Day | dose, mg | Treatments | cm³ | TGI | P value |
| P1 (T1) | D0 | 0.200 | 8 | 67.4 | 0.0 | |
| | D7 | 0.200 | | 49.5 | −26.5 | |
| | D19 | 0.200 | | 36.9 | −45.3 | 0.063 |
| | DFU | | | 13.2 | −80.4 | 0.007 |
| P1 (T2) | D0 | | 6 | 7.4 | 0.0 | |
| | D7 | | | 10.0 | 35.1 | |
| | D19 | 0.200 | | 17.8 | 140.7 | 0.179 |
| | DFU | | | 5.2 | −29.7 | 0.977 |
| P2 (T1) | D0 | 0.350 | 7 | 39.6 | 0.0 | |
| | D7 | 0.350 | | 28.6 | −27.8 | |
| | D14 | 0.350 | | 23.6 | −40.4 | 0.137 |
| | DFU | 0.350 | | 43.1 | 8.9 | 0.920 |
| P2 (T2) | D0 | 0.050 | 7 | 6.1 | 0.0 | |
| | D7 | 0.050 | | 2.1 | −64.9 | |
| | D14 | 0.050 | | 0.7 | −89.3 | 0.163 |
| | DFU | 0.050 | | 2.9 | −52.3 | 0.379 |
| P3 | D0 | 0.400 | 2 | 104.2 | 0.0 | |
| | D7 | 0.400 | | 25.2 | −75.8 | |
| | D14 | | | 24.7 | −76.3 | 0.330 |
| P4 | D0 | 0.400 | 3 | 52.7 | 0.0 | |
| | D7 | 0.400 | | 61.4 | 16.6 | |
| | D15 | 0.400 | | 51.0 | −3.1 | 0.907 |
| P5 | D0 | 0.200 | 2 | 4.3 | 0.0 | |
| | D9 | 0.200 | | 4.5 | 3.5 | |
| | D17 | | | 4.2 | −3.5 | 0.667 |

Control canine IMC patients

| | | | |
|---|---|---|---|
| P6 | D0 | 252.0 | |
| | D25 | 459.0 | 82.2 |
| P7 | D0 | 14.0 | |
| | NA | NA | |
| P8 | D0 | 32.0 | |
| | D73 | 75.3 | 124.6 |
| P9 | D0 | 30.6 | |
| | D35 | 94.6 | 209.0 |
| P10 | D0 | 9.4 | |
| | D39 | 30.8 | 228.6 |

D0, D7, D14, DFU, day 0, 7, 14 and at last follow-up (D92 in P1 and D79 in P2), respectively; T1, target tumor; T2, second tumor treated; NA, not available; Treatments refer to the total number of eCPMV injections; % TGI, percentage of tumor growth inhibition. P values obtained by regression analysis as described in Methods. The first P value shows regression analysis from D0 to D14; the second, from D0 to last follow-up.
DFU, date of follow-up; eCPMV, empty cowpea mosaic virus; NA, not available; TGI, tumor growth inhibition; Tv, tumor volume.

TABLE 3
Histopathological and immunohistochemical changes in individual tumor samples induced by eCPMV immunotherapy

| | | | | | | | | | | | |
|---|---|---|---|---|---|---|---|---|---|---|---|
| | | | | | | | | | Total | | |
| | Ki-67 | CC-3 | MPO | | | | | FoxP3/ | necrosis | NAN | nNAN |
| Patient | (%) | (%) | (%) | IL-8 | CD3* | CD20* | FoxP3* | CD3 | (%)‡ | (%) | (%) |

eCPMV-treated canine IMC patients

| | | | | | | | | | | | |
|---|---|---|---|---|---|---|---|---|---|---|---|
| P1 | | | | | | | | | | | |
| Pretreated | † | † | 0.86 | 0.00 | 241.89 | 162.53 | 63.68 | 0.26 | 0.00 | 0.00 | 0.00 |
| Post-treated (day 92) | 41.42 | 12.00 | 15.45 | 2.00 | 1422.32 | 944.63 | 133.05 | 0.09 | 5.38 | 5.19 | 0.19 |
| P2 | | | | | | | | | | | |
| Pretreated | 37.93 | 17.09 | 0.99 | 0.00 | 258.21 | 318.63 | 107.25 | 0.42 | 0.63 | 0.00 | 0.63 |
| Post-treated (day 156) | 29.48 | 5.18 | 2.09 | 2.00 | 343.89 | 80.42 | 77.51 | 0.23 | 0.97 | 0.47 | 0.50 |
| P5 | | | | | | | | | | | |
| Pretreated | 60.75 | 1.00 | 1.14 | 0.00 | 253.79 | 130.32 | 99.73 | 0.39 | 0.59 | 0.00 | 0.59 |
| Post-treated (day 17) | 38.59 | 6.41 | 9.62 | 2.00 | 759.79 | 162.74 | 88.15 | 0.12 | 2.21 | 1.43 | 0.78 |

Control canine IMC patients

| | | | | | | | | | | | |
|---|---|---|---|---|---|---|---|---|---|---|---|
| P6 | 82.24 | 8.36 | 0.56 | 0.00 | 107.16 | 160.32 | 41.47 | 0.39 | 0.04 | 0.04 | 0.00 |
| P7 | 70.70 | 3.29 | 2.50 | 0.00 | 258.21 | 145.58 | 57.88 | 0.22 | 0.01 | 0.00 | 0.01 |
| P8 | 48.88 | 3.13 | 0.26 | 1.00 | 326.00 | 307.26 | 142.74 | 0.44 | 0.00 | 0.00 | 0.00 |

*The number of CD3-positive, CD20-positive, and FoxP3-positive cells is provided per mm².
†Ki-67 and CC-3 percentages were not calculated for pretreatment biopsy of P1 due to the low availability of neoplastic cells in the pretreatment biopsy.
‡The presence of necrosis (total, neutrophil and non-neutrophil associated) was the only histopathological change assessed in H&E.CC-3, cleaved caspase-3; eCPMV, empty cowpea mosaic virus; IL-8, interleukin-8; IMC, inflammatory mammary cancer; MPO, myeloperoxidase; NAN, neutrophil-associated necrosis; nNAN, non-neutrophil-associated necrosis.

TABLE 4
Tumor changes in IMC patients by iRECIST criteria

| | | eCPMV | | Tumor | % | |
|---|---|---|---|---|---|---|
| Patient | Day | dose, mg | Treatments | size, cm | Change | iRECIST |
| | | eCPMV treated canine patients | | | | |
| P1 (T1) | D0 | 0.200 | | 11.0 | 0.0 | |
| | D7 | 0.200 | 8 | 11.0 | 0.0 | SD |
| | D19 | 0.200 | | 9.4 | −14.5 | SD |
| | DFU | | | 9.6 | −13.0 | SD |
| P1 (T2) | D0 | | | 4.1 | 0.0 | |
| | D7 | | | 5.0 | 22.0 | PD |
| | D19 | 0.200 | 6 | 5.7 | 39.0 | PD |
| | DFU | | | 9.3 | 127.6 | PD |
| P2 | D0 | 0.400 | | 15.7 | 0.0 | |
| (T1&T2)* | D7 | 0.400 | | 15.1 | −3.8 | SD |
| | D14 | 0.400 | 7 | 13.9 | −11.5 | SD |
| | DFU | 0.400 | | 19.6 | 24.8 | PD |
| P3 | D0 | 0.400 | | 19.6 | 0.0 | |
| | D7 | 0.400 | 2 | 20.2 | 3.1 | SD |
| | D14 | | | 20.1 | 2.6 | SD |
| P4 | D0 | 0.400 | | 17.3 | | |
| | D7 | 0.400 | 3 | 16.6 | −3.8 | SD |
| | D15 | 0.400 | | 17.8 | 3.4 | SD |
| P5 | D0 | 0.200 | | 3.9 | | |
| | D9 | 0.200 | 2 | 3.5 | −9.1 | SD |
| | D17 | | | 2.9 | −24.9 | SD |

Control canine IMC patients

| | | | | |
|---|---|---|---|---|
| P6 | D0 | 14.0 | | |
| | D25 | 15.9 | 13.6 | SD |
| P7 | D0 | 7.0 | | |
| | NA | NA | | |
| P8 | D0 | 4.0 | | |
| | D73 | 4.8 | 26.3 | PD |
| P9 | D0 | 5.0 | | |
| | D35 | 7.0 | 40.0 | PD |
| P10 | D0 | 3.0 | | |
| | D39 | 4.5 | 50.0 | PD |

Legends: D0, D7, D14, DFU, day 0, 7, 14, and at last follow-up (D92 in P1 and D79 in P2), respectively; T1, target tumor; T2, second tumor treated; SD, stable disease; PD, progressive disease.
*the eCPMV doses in P2 are shown as the sum for the two tumors.

TABLE 5
Lisa of monoclonal antibodies used for flow cytometry

| Antibody | Clone | Fluorochrome | Provider, cat. # |
|---|---|---|---|
| CD45 | YKIX716.13 | eFluor 450 | eBioscience ™, ThermoFisher 48-5450-41 |
| CD25 | P4A10 | Super Bright 600 | eBioscience ™, ThermoFisher, 63-0250-42 |
| CD4 | YKIX302.9 | Super Bright 645 | eBioscience ™, ThermoFisher, 64-5040-42 |
| CD8a | YCATE55.9 | Super Bright 702 | eBioscience ™, ThermoFisher, 67-5080-42 |
| EOMES | WD1928 | PerCP-eFluor 710 | eBioscience ™, ThermoFisher, 46-48877-42 |
| CD22 | RFB-4 | PE | ThermoFisher, MHCD2204 |
| FOXP3 | FJK-16s | PE-Cyanine7 | eBioscience ™, ThermoFisher, 25-5773-80 |
| MHC Class II | YKIX334.2 | APC | ThermoFisher, 17-5909-42 |
| Ki-67 | SolA15 | Alexa Fluor 700 | eBioscience ™, ThermoFisher, 56-5698-82 |
| CD5 | YKIX322.3 | APC-eFluor 780 | eBioscience ™, ThermoFisher, 47-5050-42 |
| CD14 | M5E2 | Brilliant Violet 785 | Biolegend, 301840 |
| CD3 | CA17.2A12 | FITC | BioRad, MCA1774F |
| Granzyme B | Granzyme B | PE-CF594 | BD, 562462 |
| Dead cells | | Zombie Aqua ™ Fixable Viability Kit | Biolegend, 423102 |

TABLE 6
Blood cell, biochemistry and cytokine changes during eCPMV immunotherapy in canine IMC patients.

| Variable | Day 0 | Day 7 | Day 14 | P value * |
|---|---|---|---|---|
| Total leukocytes (cells/μL) | 12452.0 ± 1303.0 | 9242.0 ± 1414.1 | 13920.0 ± 3417.4 | 0.043; 0.625; 0.080 |
| Total lymphocytes (cells/μL) | 1144.6 ± 334.7 | 925.4 ± 217.5 | 1438.4 ± 298.9 | 0.576; 0.536; 0.097 |
| Total monocytes (cells/μL) | 1073.6 ± 85.3 | 896.8 ± 172.6 | 747.0 ± 253.1 | 0.202; 0.242; 0.382 |
| Total mature neutrophils (cells/μL) | 9902.8 ± 1144.6 | 6894.8 ± 1021.2 | 11099.4 ± 2979.8 | 0.043; 0.686; 0.080 |
| Total immature neutrophils (cells/μL) | 0.0 | 84.8 ± 29.2 | 448.4 ± 212.3 | 0.068; 0.043; 0.166 |
| Total proteins (g/dL) | 7.4 ± 0.4 | 7.3 ± 0.4 | 6.4 ± 0.4 | 0.655; 0.273; 0.655 |
| Interleukin-8 (pg/mL) | 1366.5 ± 798.9 | 4296.9 ± 1055.9 | 2036.8 ± 1496.2 | 0.068; 0.593; 0.285 |

Legends: ± denotes standard error;
*P values estimated by Student-T test or Wilcoxon test for day 7 and day 14 compared to day 0, and between day 7 and 14, respectively.

TABLE 7
Blood cell and protein changes in untreated
control canine IMC patients

| | | | P |
|---|---|---|---|
| Variable | Day 0 | Day 14 | value* |
| Total leukocytes (cells/μL) | 7860.0 ± 599.7 | 7333.3 ± 1975.1 | 0.593 |
| Total | 1530.0 ± 387.7 | 861.3 ± 214.8 | 0.109 |
| lymphocytes (cells/μL) | | | |
| Total monocytes (cells/μL) | 292.4 ± 78.4 | 658.7 ± 513.1 | 0.593 |
| Total mature | 5664.8 ± 241.2 | 5555.3 ± 1341.3 | 0.593 |
| neutrophils (cells/μL) | | | |
| Total immature | 40.8 ± 26.5 | 54.0 ± 29.1 | 0.593 |
| neutrophils (cells/μL) | | | |
| Total proteins (g/dL) | 6.4 ± 0.3 | 5.8 ± 1.2 | 0.655 |

Legends: ± denotes standard error;
*P values estimated by Student-T test or Wilcoxon test.

TABLE 8
Changes in the percentage of CD8, Treg and
GZMB cells during eCPMV immunotherapy

| | | | First isolation |
|---|---|---|---|
| Variable | Patient | Day 0 | after treatment |
| CD8⁺ cells | P1 | 25.80 | 37.30 |
| | P2 | 44.30 | 34.10 |
| | P4 | 46.00 | 46.30 |
| | P5 | 45.70 | 49.80 |
| FoxP3⁺ (Treg) cells | P1 | 19.40 | 21.30 |
| | P2 | 13.50 | 9.39 |
| | P4 | 15.10 | 13.30 |
| | P5 | 12.00 | 10.70 |
| Treg⁺/CD8⁺ cells ratio | P1 | 0.75 | 0.57 |
| | P2 | 0.30 | 0.28 |
| | P4 | 0.33 | 0.29 |
| | P5 | 0.26 | 0.21 |
| CD8⁺ GZMB⁺ cells | P1 | 28.90 | 30.30 |
| | P2 | 77.90 | 59.60 |
| | P4 | 22.40 | 14.40 |
| | P5 | 69.80 | 63.40 |

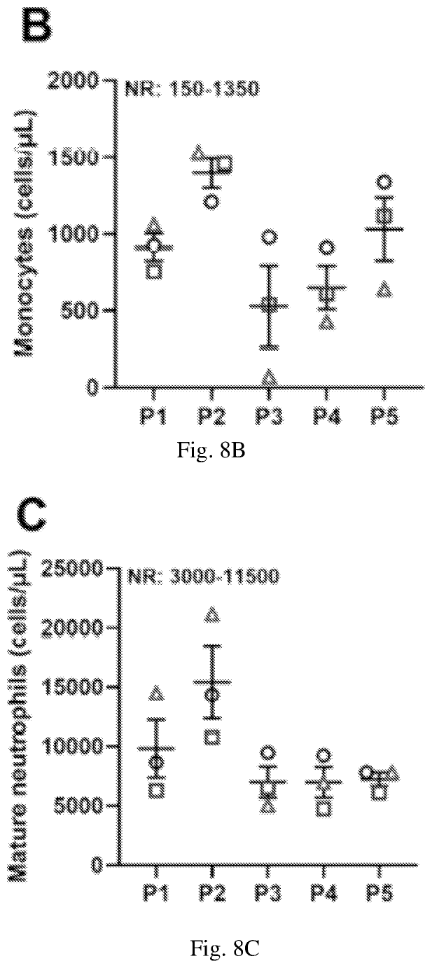

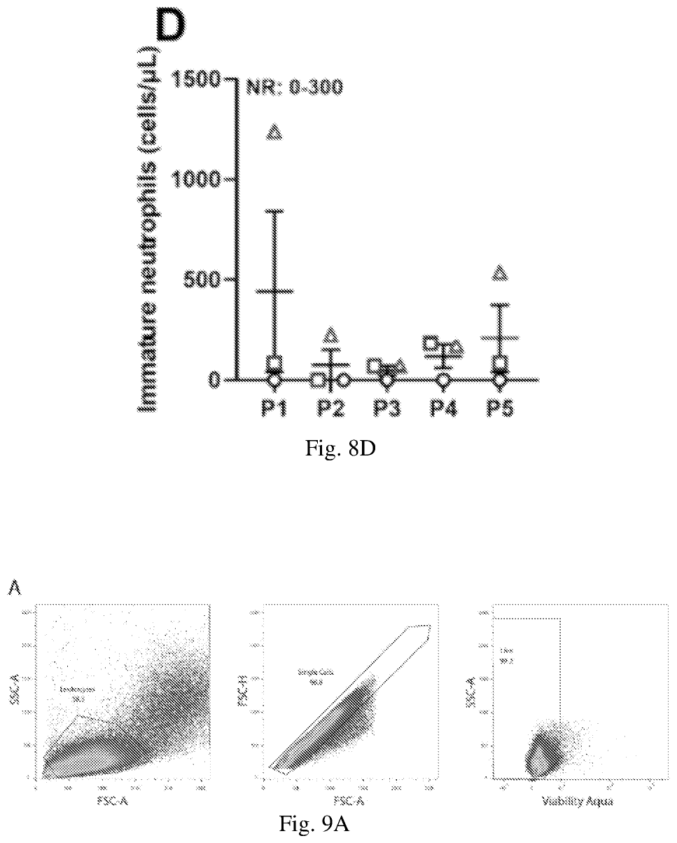

Legends: GZMB: granzyme B; See FIG. 8 for gating information.

TABLE 9
Histopathological and Immunohistochemical changes induced
in tumor samples by neoadjuvant in situ eCPMV therapy

| | Pre- | Post- | |
|---|---|---|---|
| | treatment | treatment | P |
| Markers | n = 6 | n = 3 | value* |
| Total necrosis (%) ^{a} | 0.21 ± 0.31 | 2.85 ± 2.27 | 0.024* |
| Neutrophil-associated | 0.01 ± 0.02 | 2.36 ± 2.49 | 0.024* |
| necrosis (%) | | | |
| Non-neutrophil- | | | |
| associated necrosis | | | |
| (%) | 0.21 ± 0.31 | 0.49 ± 0.30 | 0.262* |
| MPO (%) | 1.1 ± 0.4 | 9.59 ± 3.9 | 0.032** |
| IL-8 score | | | 0.011 |
| Absence (0) | 5 | 0 | |
| Low (1+) | 1 | 0 | |
| Intermediate (2+) | 0 | 3 | |
| CD3⁺ per mm² | 240.9 ± 29.4 | 842.0 ± 314.0 | 0.024* |
| CD20⁺ per mm² | 204.1 ± 34.8 | 395.9 ± 275.4 | 0.999* |
| FoxP3⁺ per mm² | 85.5 ± 15.4 | 99.6 ± 17.0 | 0.592** |
| FoxP3⁺/CD3⁺ ratio | 0.35 ± 0.04 | 0.15 ± 0.04 | 0.012** |
| Ki-67⁺ (%) | 60.1 ± 7.8 ^{b} | 36.5 ± 3.6 | 0.038** |
| CC-3⁺ (%) | 6.6 ± 2.9 ^{b} | 7.9 ± 2.1 | 0.767** |

Legends: ± denotes standard error; CC3: cleaved Caspase-3; MPO: myeloperoxidase; IL- 8: interleukin-8;
*P values estimated by Mann-Whitney U test (*), Student-T test (**), and Chi-square test.
^{a} the presence of necrosis (total, neutrophil- and non-neutrophil- associated) was the only histopathological change assessed in hematoxylin & eosin.
^{b} Pre- treatment biopsy of P1 was not included for Ki-67 and CC-3 quantification due to low availability of neoplastic cells.

### Hematological, Flow Cytometry, Biochemical and Cytokine Analyses

A blood sample (˜10 mL) was collected from each patient at D0, D7, and D14 to evaluate changes induced by eCPMV immunotherapy. Hematological analyses were done using a standard hematology analyzer (ADVIA 120, Siemens Health-care, Madrid, Spain). Samples collected at D0, D14, and at various time points after surgery were used to isolate peripheral blood mononuclear cells (PBMCs) for flow cytometry using the Lymphocyte Isolation Reagent kit per manufacturer's instructions (Ficoll 1.077 g/mL solution, Rafer, Zaragoza, Spain). Isolated cells were transferred to a freezing medium (70% RPMI, 20% DMSO and 10% fetal bovine serum), frozen at −80° C. overnight, and then transferred into liquid nitrogen until sample processing. Flow cytometry analysis was performed using a 14-color panel (Table 5) using reagents and procedures as previously described. Stained samples were acquired using LSR Fortessa II (BD Biosciences, San Jose, California, USA) equipped with 4 lasers and 16 detectors. Flow cytometric analysis was performed using FlowJo software (V.10.7.1; BD Bioscience).

The biochemical panel (glucose, creatinine, urea and alanine aminotransferase (ALT)) of each patient was performed by reflection spectrophotometry (Refrovet Plus, Scil animal care company, Viernhheim, Germany) and for total proteins by Biuret's colorimetric test (Brad-ford Diagnostics, Sigma-Aldrich, St. Louis, MO, USA). Cytokine measurement in plasma samples was performed using the MILLIPLEX Canine Cytokine/Chemokine Magnetic Bead Panel (Merck Millipore, Burlington, MA, USA). The evaluation of hematological, biochemical and other adverse events related to eCPMV immunotherapy was performed according to the Veterinary Cooperative Oncology Group criteria in the V.2.

### Histopathology and Immunohistochemistry Assays

Single 3 μm tumor tissue sections were used for histopathology and

immunohistochemistry (IHC). The IHC assays for Ki-67, CC-3, myeloperoxidase (MPO), IL-8, CD3, CD20, and FoxP3, and the scoring of all markers are reported following REMARK guidelines.

### Statistical Analyses

Primary outcomes were efficacy, measured by reduction in Tv; biosafety, measured by evaluation of hematological and biochemistry changes in blood and plasma; and, survival of treated patients, measured by tracking clinical status of patients. The Kaplan-Meier method with the log-rank test was used to estimate survival. Unpaired Student's t-test, Mann-Whitney U test, Fisher's exact test, and the χ2 test were used as appropriate. Two-tailed p values less than 0.05 were considered statistically significant. Statistical analyses were carried out using IBM SPSS Statistics program (V.25) and GraphPad Prism (V.7.02; GraphPad San Diego, California, USA) software.

### Canine Patient Recruitment and Selection Criteria

Given the rarity of IMC and the scarcity of specific beneficial therapies, any female canine patients diagnosed with IMC were recruited. Ten IMC patients were enrolled in this study (Table 1). Two clinical presentations of IMC have been recognized: primary IMC spontaneously occurs in dogs without any previous mammary nodules; secondary IMC arises in patients with a history of mammary tumors that develop inflammatory signs. One dog was diagnosed with a primary IMC and nine dogs were diagnosed with secondary IMC (Table 1). Inclusion criteria at diagnosis included female dogs without evidence of metastatic dissemination, no severe infection of the target tumor, no chronic-life threatening disease or any systemic disease that could influence the immune system response (such as endocrinopathies, immune-mediated disease, leishmaniasis and ehrlichiosis), and no treatment with immunosuppressive drugs. The histopathological classification of tumors was performed according to the veterinary histological classification, and the histological grade of malignancy was performed as previously described.

### Collection of Tumor Biopsies and Surgical Procedures

For the collection of the incisional tumor biopsy, all patients were sedated (Medetomidine, methadone (10 and 300 micrograms/kg, intramuscularly, respectively)) when the incisional biopsy was collected and, when necessary, analgesia was provided (Tramadol, 2 mg/kg, orally, every 12 h for 4 days). If dogs were not under sedation, the intratumoral eCPMV inoculation was performed under topical local anesthesia using a tetracaine ointment.

For surgical procedures, all patients were premedicated with medetomidine and methadone (10 and 300 micrograms/kg, intramuscularly, respectively), followed by induction with propofol (1 mg/kg, intravenously) and inhalational anesthesia with isoflurane (1.5%-2.5%). Intravenous cephazolin was given 20 min before surgery (22 mg/kg). Further, depending on the mastectomy procedure, transversus abdominis plane block with bupivacaine (up to 2 mg/kg) and/or epidural anesthesia using morphine 0.1 mg/kg plus bupivacaine (up to 2 mg/kg) was provided. Diffusion catheters were placed during surgery (DC Mila International Inc®) in order to administer bupivacaine (1-2 mg/kg every 6 h) in the post-operative period. Soft sterile wound dressings and a tubular mesh were placed to cover the wound. Post-surgical therapy also included firocoxib (5 mg/kg, orally every 12 h for 7 days) and tramadol (3 mg/kg, orally every 12 h for 3 days). No post-operative antibiotics were prescribed. Catheters were left in place for 3 days. Wounds healed uneventfully and skin sutures were removed after 12 days.

eCPMV Nanoparticles and Dosage

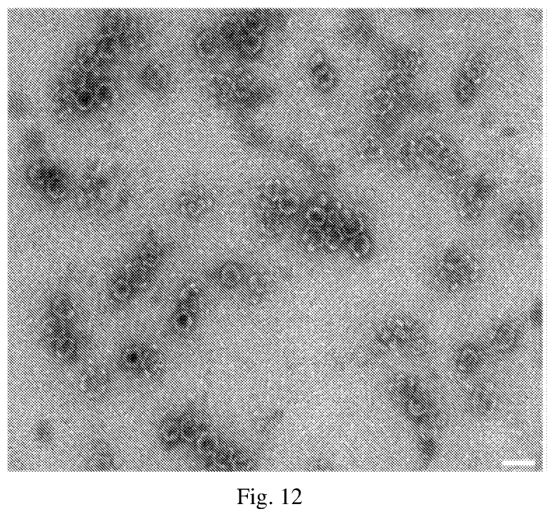

CPMV particles devoid of RNA1 or RNA2 (empty CPMV or eCPMV) were produced through agroinfiltration of Nicotiana benthamiana plants using vector PEAQexpress-VP60-24K. For eCPMV purification, infiltrated leaves were harvested 7 days post infiltration and homogenized with 2-3 vol of 0.1 M potassium phosphate (KP) buffer (pH 7.0) followed by addition of ¼ vol of a 20% PEG solution in water with 1 M NaCl to the clarified homogenate; PEG precipitation was carried out by stirring overnight at 4° C. After 2× centrifugation (27,000 g, 15 mins, at 4° C.) the pellet was resuspended in 0.01 M KP buffer and ultracentrifuged (130,000 g, 3 hours, at 4° C.) over a 30% sucrose cushion. The sucrose fraction was collected, dialyzed against 0.1 M KP buffer, and characterized using size exclusion chromatography, native and denaturing gel electrophoresis, ultraviolet-visible spectroscopy, and transmission electron microscopy (FIG. 12).

eCPMV doses were based on a previously published study in canine oral melanoma patients. Briefly, the eCPMV nanoparticles were diluted in 0.5 ml of sterile phosphate buffered saline (PBS) and injected using a 25G needle (FIG. 5). The injected PBS volume was equally distributed in 3 to 5 locations within a treated tumor. The total volume of PBS was equally distributed when applying injections. The amount of eCPMV per treatment was constant regardless of the tumor size.

### Medical Therapy

The medical therapy consists of a cyclooxygenase-2 (COX-2) inhibitor (firocoxib, 5 mg/kg; daily; oral), cyclophosphamide-based metronomic chemotherapy (12.5 mg/m²; daily; oral) and toceranib phosphate (at 2.4-2.7 mg/kg/oral 3 days per week).

### Histopathological Assesment of Tumor Necrosis Area

Hematoxylin and eosin (H&E)-stained tumor tissue sections were used for histopathological diagnosis and the assesment of tumor necrosis area. Tumor necrosis area was evaluated by CellSense Entry software (Olympus, Waltham, MA, USA) measuring areas of neutrophil-associated necrosis and non-neutrophil-associated necrosis on whole microscopic slides, and calculating the percentage of necrosis as necrosis area/total tumor area*100. Total necrosis was defined as the summatory of neutrophil-associated necrosis and non-neutrophil-associated necrosis.

### Immunohistochemistry (IHC) Assays

Tumor biopsies from P1, P2, and P5 (pre-and post-treatment) and three control dogs were available for IHC. Unfortunately, the pre-treatment biopsy from P1 consists mostly of skin tissue with a few tumor cells and immune cells (˜5% and ˜20% of tumor and immune cells, respectively); P2 post-treatment biopsy was taken at necropsy 24 h after the dog died, affecting the quality of collected tumor samples. Histopathologic analysis demonstrated good tissue quality in the necropsy tissue. P3 and P4 patients were referred to our Hospital with documented IMC diagnosis, undergone a pre-treatment biopsy (not available to us), and they died outside our Hospital without tumor biopsy collection. Single 3 μm tumor tissue cuts were used for the immunostaining of Ki-67, Cleaved Caspase-3 (CC3), myeloperoxidase (MPO), and interleukin-8 (IL-8). Deparaffination and antigen retrieval were performed in a PT Lab Vision module (Thermo Fisher Scientific Inc, Waltham, MA, USA) by immersion in 1 mM EDTA buffered solution at 95° C. for 20 minutes, the sections were cooled down, and immunolabelled in an automatic autostainer (Autostainer 480S, Thermo Fisher), using a polymer-based method and a peroxidase detection system (Ultra Vision Quanto MAD-021881QK, Master diagnostic, Granada, Spain). Antibodies against Ki-67 (Master Diagnóstica, Sevilla, Spain; Cat. No. MAD-000310QD ready-to-use; clone SP6), CC3 (Cell Signaling, Danvers, Ma, USA; Cat. No. 9661L; 4 μg/ml), MPO (Dako, Santa Clara, Ca, USA; Cat. No. A0398; 33 μg/ml), IL-8 (Abcam, Cambridge, UK; Cat. No. Ab106350; 2 μg/ml), CD3 (Dako, Santa Clara, Ca, USA; Cat. No. A045201; 6 μg/ml), CD20 (Thermo fisher scientific, Walham, Ma, USA; Cat. No. RB-9013; 0.33 μg/ml), FoxP3 (Master Diagnóstica, Sevilla, Spain; Cat. No. MAD-000536QD ready-to-use; clone SP97) were used as recommended by manufacturers. External controls (canine thymus for CC-3 and FoxP3; neutrophilic sebaceous adenitis for MPO and IL-8; canine lymph node for CD3 and CD20) and internal controls (tumor mitotic figures for Ki-67) were used. The corresponding negative control slides were obtained by replacing the primary antibody with a nonreacting antibody on canine tissues.

### Scoring of IHC Markers

Proliferation and apoptosis indexes were defined as the percentage of positive tumor cells with the Ki-67 and CC3 markers, respectively, by counting positive and negative nuclei in 10 high-power-fields (40×). MPO was quantitatively evaluated with the binary image thresholding method as the percentage of positive area in ten 100× fields. IL-8 was semi-quantitatively scored as negative (0), low (1+), moderate 2+ and strong (3+) extracellular stromal immunolabeling. The number of B (CD20⁺), T (CD3⁺), and T regulatory lymphocytes (FoxP3⁺) was assessed in hot spots (lymphocytic-rich areas) as the number of positive cells/mm². T regulatory lymphocytes/T lymphocyte ratio was calculated dividing the number of FoxP3⁺ cells/mm² by the number of CD3⁺ cells/mm².

### Cytokine Measurement

The MILLIPLEX® Canine Cytokine/Chemokine Magnetic Bead Panel was used to measure 13 cytokines in plasma samples as indicted by the manufacturer (Merck Millipore, Burlington, MA, USA): GM-CSF, IFN-γ, KC (CXCL1), IP-10 (CXCL10), IL-2, IL-6, IL-7, IL-8, IL-10, IL-15, IL-18, MCP-1 (CCL2), and TNF-α.

### Statistical Analyses

Primary outcomes were efficacy, measured by reduction in tumor volume, biosafety by evaluation of hematological and biochemistry changes in blood and plasma, and survival of treated patients by tracking physical status of patients. For evaluation of individual eCPMV-induced changes in tumor size between start of treatment and after the first and second eCPMV injections, linear regression analysis of percentage of changes in tumor volumes by days for each tumor was performed. To evaluate potential toxic and immunological effects of eCPMV therapy in dogs, a two-tailed Student's t-test or Wilcoxon test were performed as appropriate to compare eCPMV-induced changes in blood cell numbers and plasma levels of total proteins (albumin and globulins), glucose, urea, creatinine, and ALT, and cytokine levels in samples collected before and during treatment, and at surgery. Individual changes in blood parameters and IL-8 levels were analyzed by linear regression analysis. Survival was calculated from the date of diagnosis with death from mammary cancer scored as an event and censoring of other patients at the date of last follow-up or non-disease-related death. The Kaplan-Meier method with the log-rank test was used to estimate survival. The presence of necrosis and the immunolabeling of the IHC markers were compared between the pre-treatment/control and post-treatment biopsies to determine the effect of the eCPMV on the tumor tissue. Unpaired Student t-test or Mann-Whitney U test was used to compare continuous variables between groups. The Fisher's exact test and the Chi-square test were used to compare binary categorical variables and categorical variables with more than two categories, respectively. Pearson's correlation coefficient was used to evaluate the correlation between continuous variables. Two-tailed P values less than 0.05 were considered statistically significant. Statistical analyses were carried out using IBM SPSS Statistics program (version v.25; Armonk, NY, USA) and GraphPad Prism (version 7.02; GraphPad San Diego, CA, USA) software

### Results

eCPMV Immunotherapy Vaccination Induces Tumor Shrinkage in IMC Patients

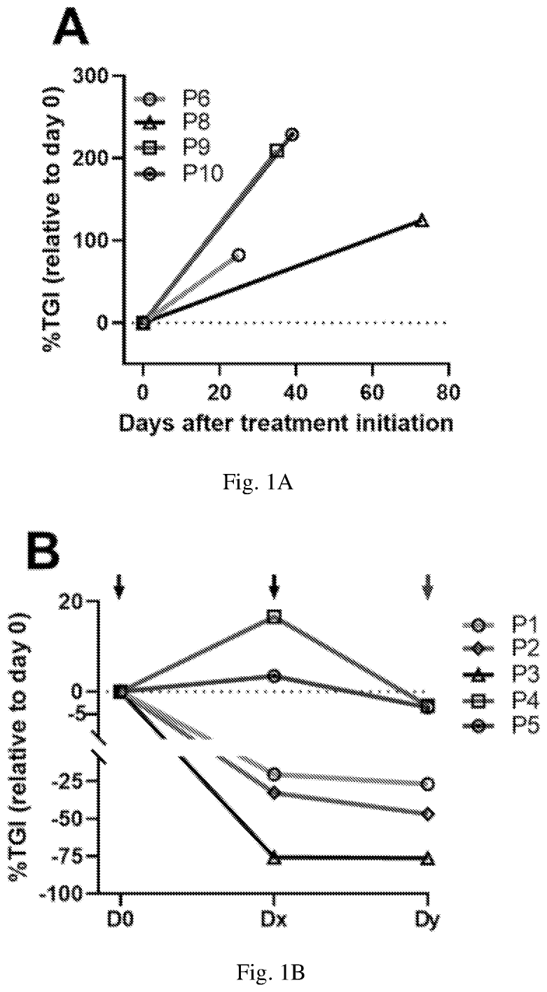

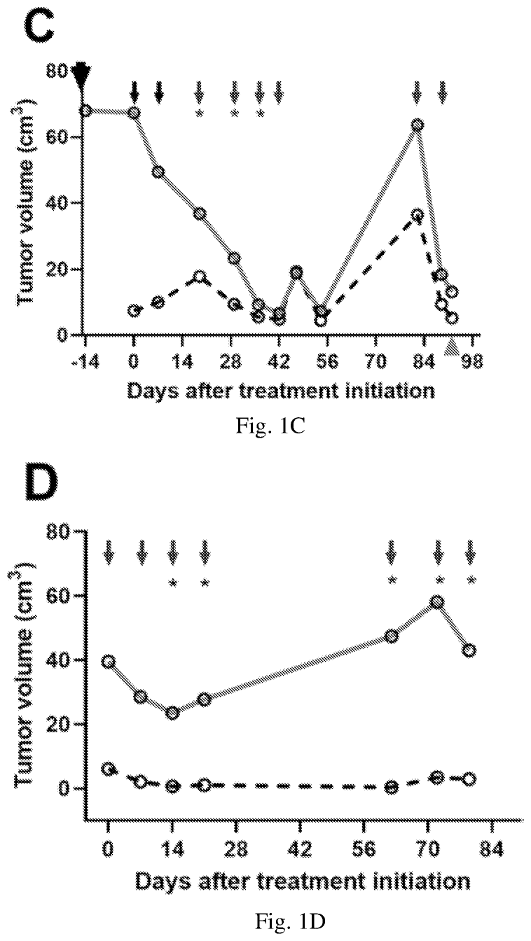

Of the 10 enrolled IMC patients, 5 did not receive the eCPMV immunotherapy because the owners declined treatment. These patients were used as control cases (FIG. 1A; Table 2). The remaining five IMC patients were treated with eCPMV immunotherapy: Patients P3 and P5 received two injections, P4, three injections (FIG. 1B), P1, eight injections (FIG. 1C), and P2, seven injections (FIG. 1D). Owners of patients P3 and P4 refused additional treatment for personal reasons. P3 died at D109 of unknown cause and P4 was euthanized at D165 due to disease progression (Table 1). P1 and P2 are described in detail below. Treatment response evaluated by % TGI demonstrated shrinkage of Tv at D7 in three patients (P1, P2, and P3, FIG. 1B and Table 2) and tumor growth (pseudoprogression) in patients P4 and P5 with tumor shrinkage by D14 (FIG. 1B and Table 2). The eCPMV immunotherapy resulted in tumor reduction sufficient to enable surgery in P5 and P1 after 2 and 8 injections, respectively (FIG. 1B,C). Regression analyses indicate that all patients had a reduction in tumor burden after two eCPMV injections (Table 2).

Patients P1 and P2 received more treatments and were followed up in more detail. P1 presented two concurrent large masses classified as special type carcinoma located in different mammary chains (Table 2). Before eCPMV treatment, this patient was on anti-COX-2 (firocoxib) therapy for 15 days without responding to that therapy. As shown in FIG. 1C, the tumor growth kinetics demonstrate that eCPMV immunotherapy resulted in an immediate response that was sustained following each weekly eCPMV injection. At D20, medical therapy was added for 3 weeks to the eCPMV immunotherapy, but due to toxicities, toceranib was removed (at D42, after 23 days of toceranib treatment), and the dog received only firocoxib and metronomic cyclophosphamide along with eCPMV immunotherapy for three additional treatments. With regards to durability of treatment response, a rapid tumor growth was observed during the time the patient was receiving firocoxib and metronomic cyclo-phosphamide, but not eCPMV immunotherapy because the owners and the dog were not available (D54-D82 in FIG. 1C). However, as soon as the eCPMV injections were resumed, a sharp decrease in tumor growth was observed to the point that the patient underwent bilateral radical mastectomy to remove the tumor masses (D92 in FIG. 1C). Of note, treatment of a second IMC mass was also started on D19 and the clinical response was similar to the largest mass treated at the same time (FIG. 1B). This patient lived ˜6 months and died of noncancer-related renal failure.

P2 with two tumor masses classified as simple carcinomas in different mammary chains was enrolled from an external clinic at D57 of her diagnosis. For simplicity, the first eCPMV injection is represented here as D0. This tumor showed fluctuation in the response to eCPMV immunotherapy with a decrease in tumor size followed by tumor growth up to D70, and again, a decrease in tumor size up to D79 (FIG. 1D). Shortly after that visit the patient was euthanized in a different clinic at about D99 due to dyspnea and metastatic lung disease. Similar to patient P1, tumor growth was observed during the time the patient was not on treatment because the Hospital was closed due to COVID-19 lockdown (D21 to D62 in FIG. 1D). Interestingly, a second eCPMV-treated mass in this patient was more responsive to the therapy and after a decrease in the Tv, it never significantly grew again and remained almost unchanged during the observation period (FIG. 1D; Table 2). This patient lived ˜5 months.

eCPMV Immunotherapy is Not Toxic

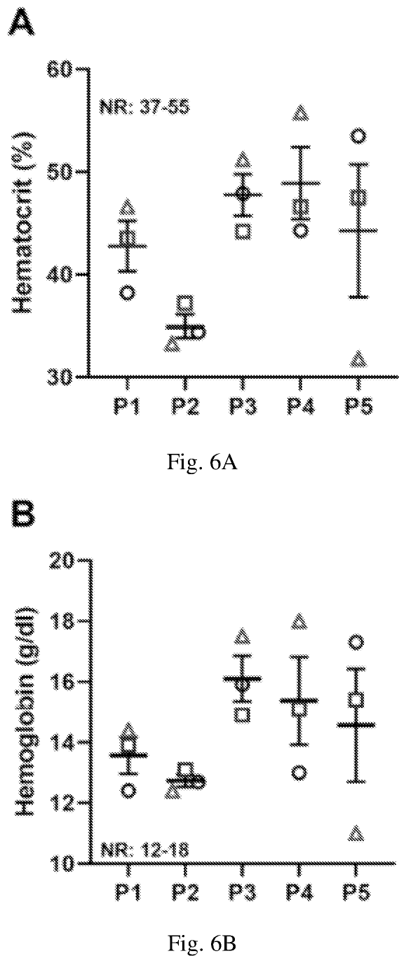

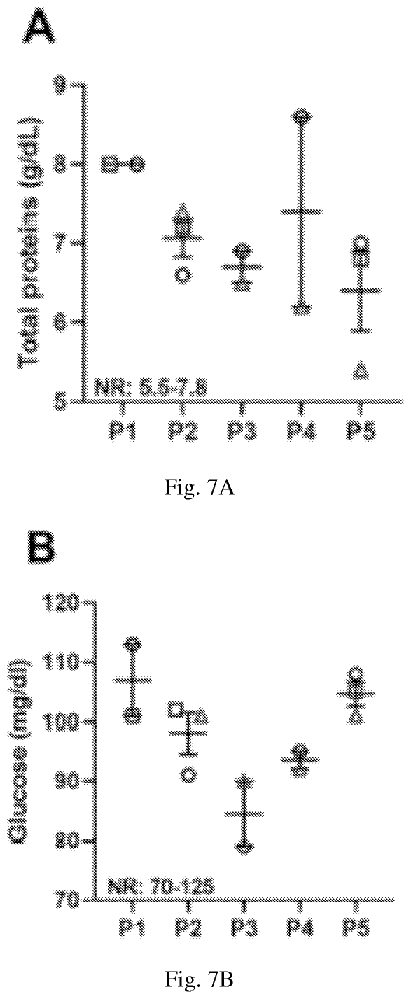

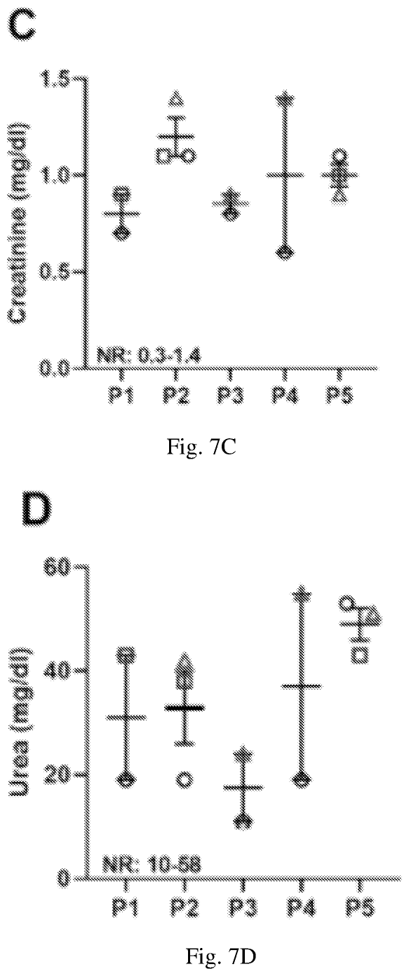

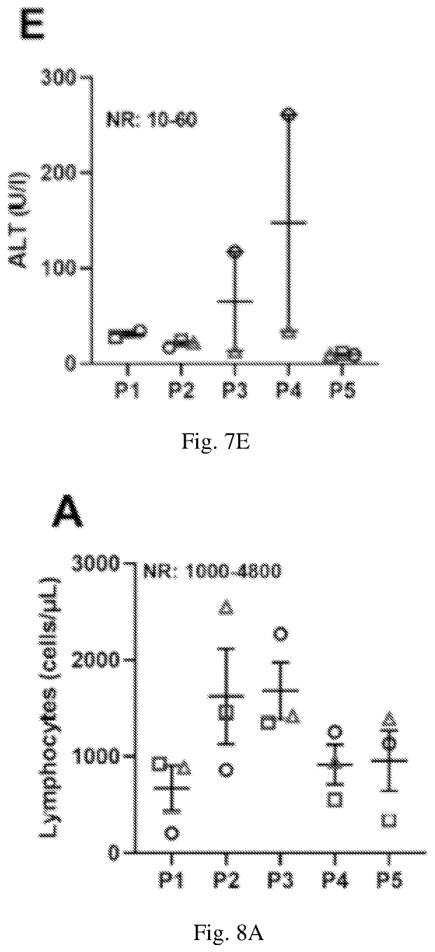

No adverse reactions at injection site or systemic reactions were observed during the 4-hour period after each eCPMV administration. eCPMV immunotherapy did not induce significant fluctuations in hematocrit and hemoglobin levels in any dog over the 14-day period. Fluctuations remained within the normal range in all eCPMV treated dogs (FIG. 6). Further, no significant changes in total proteins (albumin and globulins levels) were observed during the treatment period (online supplemental Table 6 and FIG. 7A). Despite fluctuations in the levels of glucose, urea, creatinine, and ALT during eCPMV immunotherapy, these changes remained within the normal ranges (FIG. 7B-E). Hence, these findings indicate that eCPMV immunotherapy with this dosing does not negatively affect hepatic, renal and digestive functions in the vaccinated dogs. No changes were observed in blood cell numbers, and protein levels in the control patients not treated with eCPMV (Table 7).

Per our QOL questionnaire, QOL was improved in three dogs (P1, P2, and P3) and two dogs reported no changes in QOL during the 14-day observation time (P4 and P5; data not shown).

eCPMV Immunotherapy Induces Changes in Immune Blood Cell Populations

White blood cell analysis indicates a decrease in lymphocyte, monocyte, and mature neutrophil numbers at D7 in three dogs (P3, P4, and P5), with a subsequent increase close to pretreatment levels in lymphocytes and mature neutrophils, and a further decrease in the monocyte levels (FIG. 8A-C). Immature neutrophils in these three cases remain close to or slightly higher than pretreatment levels (FIG. 8D). In the other two eCPMV-treated dogs (P1 and P2), lymphocytes and monocytes remained increased at D14. An increase in mature and immature neutrophils was observed in both dogs, with P1 showing a large increase in immature neutrophils without reaching statistical significance (FIG. 8D). Change in blood cell numbers during treatment in all treated dogs is shown in Table 6. These findings suggest that eCPMV immunotherapy induces an increase in peripheral blood inflammatory cells.

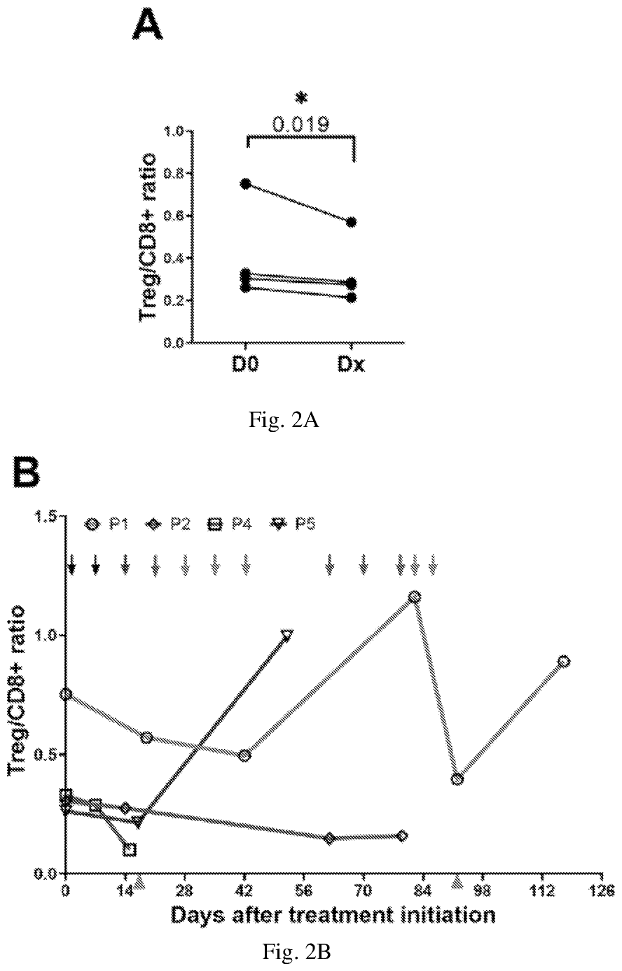

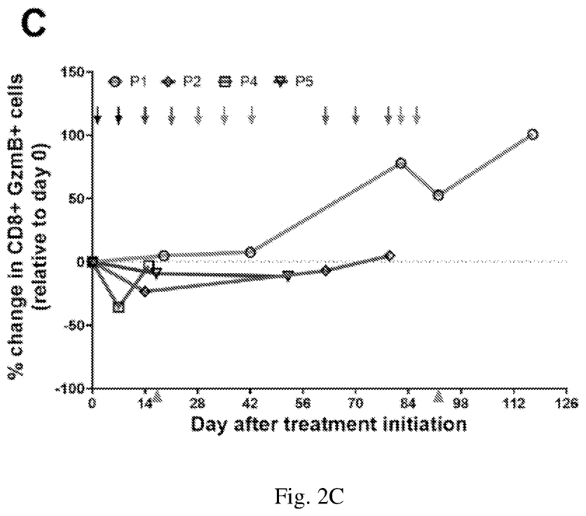

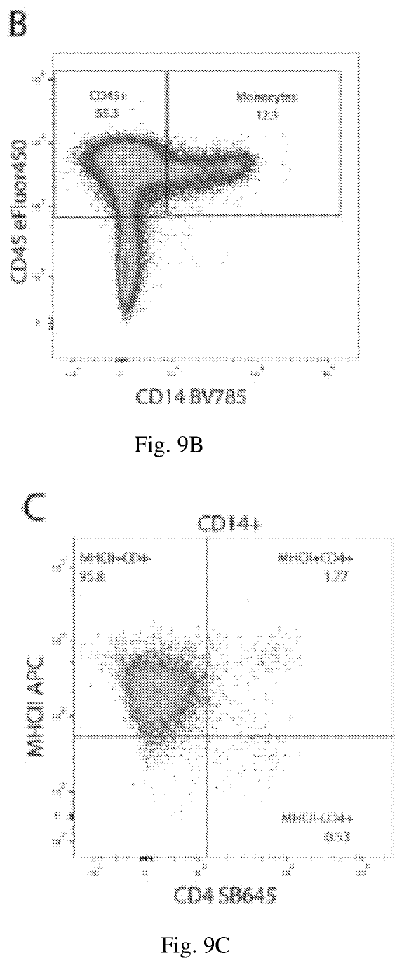

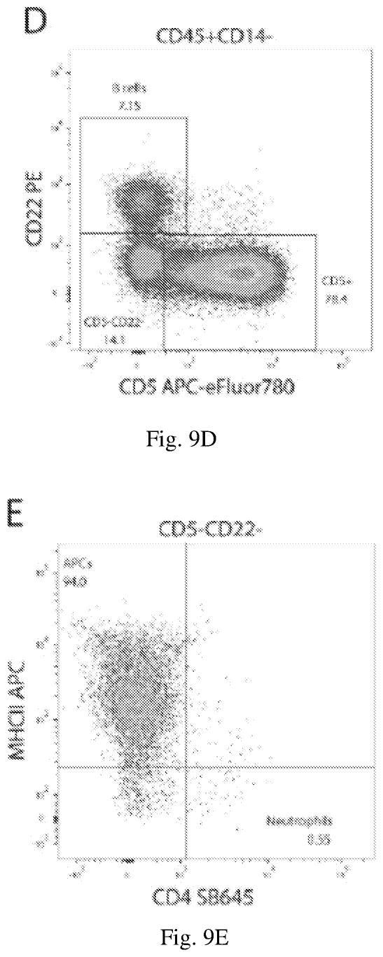

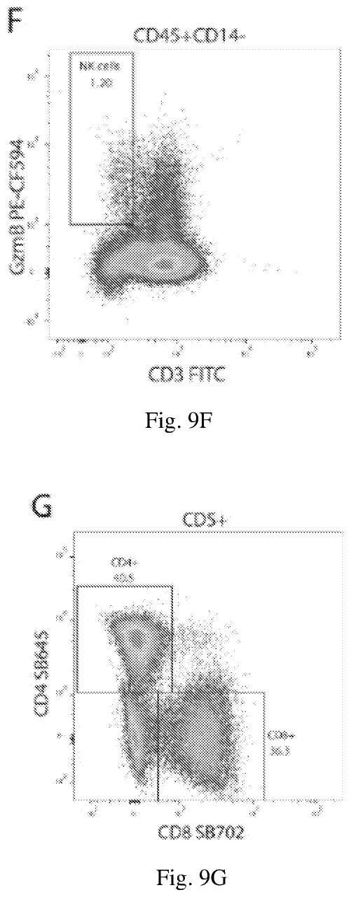

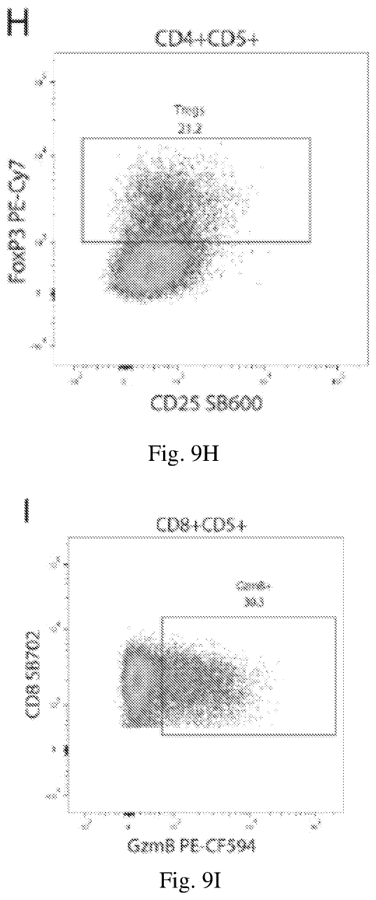

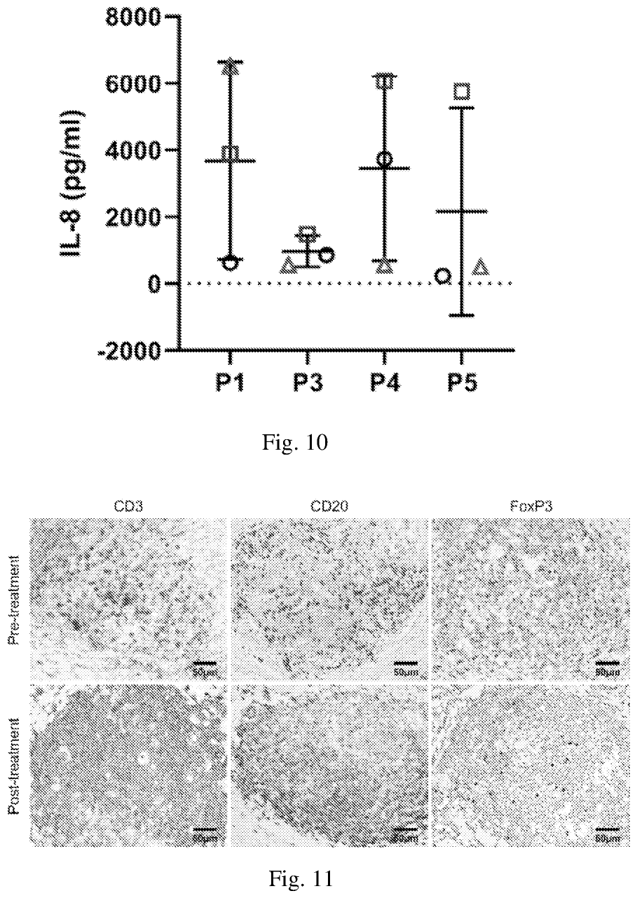

Immunophenotyping by flow cytometry on canine PBMCs collected at D0 and after eCPMV immunotherapy detected a decrease of the regulatory T cells (Treg)/CD8⁺ ratio in treated IMC patients to various degrees (FIG. 2A; p=0.019; gating strategy is shown in FIG. 9). Individual changes in CD8⁺, Treg⁺, Treg⁺/CD8⁺ cells ratio, and CD8⁺ granzyme B (GZMB)⁺ cells numbers is presented in Table 8. Individual analysis of immune cell changes in patients P1, P2, P4, and P5 over time during eCPMV immunotherapy revealed a general trend of decreased Treg⁺/CD8⁺ ratio in the various patients with some fluctuations in P1 and P5 (FIG. 2B). Of note, as we observed for the % TGI, an increase in Treg⁺/CD8⁺ ratio was observed in P1 around the time when eCPMV immunotherapy was interrupted (D54 to D82 in FIG. 2B). However, as soon as the treatment was restarted at D82, a subsequent sharp decrease in Treg⁺/CD8⁺ ratio was observed in P1 (FIG. 2B). In addition, a sharp increase in Treg⁺/CD8⁺ ratio post-surgery was observed in P1 and P5 patients, the only two dogs that underwent surgery (FIG. 2B).

Changes in CD8⁺ T cells expressing cytotoxicity marker GZMB were also evaluated in four patients (FIG. 2C and Table 8). A fluctuation in CD8⁺GZMB⁺ T cells was observed in the treated IMC patients, with an initial decrease in circulating CD8⁺GZMB⁺ T cells in patients P2, P4, and P5 and a subsequent increase in patients P2 and P5. Patient P1 showed increases in CD8⁺GZMB⁺ T cells from the start of eCPMV immunotherapy up to D84 when the levels started decreasing until D92 when surgery was performed. Then, a sharp postsurgery increase in CD8⁺GZMB⁺ T cells was observed until the last follow-up on D117 (FIG. 2C). This increase pattern was significant over the treatment period (FIG. 2C; p=0.007 (data not shown)).

eCPMV Immunotherapy Induces Changes in IL-8 Plasma Levels

Of the 13 cytokines analyzed, no significant eCPMV-induced changes were observed at D7 and D14 when compared with D0, except for IL-8 which increased at D7 in P1, P4, and P5. The IL-8 levels decreased by D14 in all dogs, except in P1 where the levels showed a 10-fold increase when compared with pretreatment levels (FIG. 9; P=0.039). Average changes in IL-8 plasma levels in all patients during treatment are shown in Table 6.

Neoadjuvant in situ eCPMV Immunotherapy is Associated With a Strong Neutrophilic Infiltration and Tumor Cell Death in Tumor Tissues and Tumor Emboli

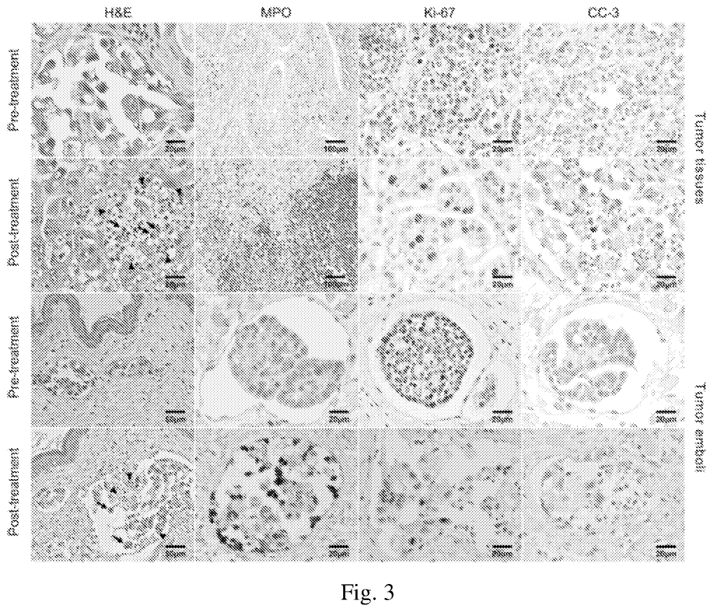

eCPMV immunotherapy induced a large neutrophilic infiltration and associated tumor cell death (necrosis) in post-treatment as compared with pretreatment tumor samples (Table 3; FIG. 3; H&E staining) and emboli (FIG. 3; H&E staining). The percentage of total necrosis area and neutrophil-associated necrosis area was significantly higher in post-treatment as compared with pretreatment tumor tissues (p=0.024, for both; Table 9; Table 3). Furthermore, MPO expression, an enzyme secreted by neutrophils during inflammation, was significantly higher in post-treatment than in pretreatment tumor specimens (p=0.032; Table 9; FIG. 3; Table 3). In addition, tumor Ki-67 proliferation index (PI) was lower in post-treatment than in pretreatment tumor samples (FIG. 3; Table 3; Table 9; P=0.038). The percentage of tumor cells undergoing apoptosis (CC-3⁺) was not different between pretreatment and post-treatment tumor samples (FIG. 3; Table 3; Table 9), indicating that apoptosis is not the mechanism of tumor cell death. Of note, although it was not possible to quantify the CC-3 expression in tumor emboli due to insufficient number of cells in emboli to apply the scoring system, a similar pattern of neutrophil influx, changes in MPO, CC-3, and Ki-67 immunostaining as in the tumor tissue were observed in tumor emboli from post-treatment samples as compared with pretreatment samples (FIG. 3). Further, a significant increase in CD3⁺ T lymphocyte density was observed in post-treatment compared with pretreatment samples (p=0.024; Table 9; Table 3; FIG. 11). An increase in CD20⁺ B lymphocytes in post-treatment was observed in two of three IMC patients (Table 3; Table 9 and FIG. 11), and a decrease in FoxP3⁺ lymphocytes was observed in two of three post-treatment samples (Table 3; Table 9 and FIG. 11). In addition, the FoxP3⁺/CD3⁺ ratio decreased significantly in post-treatment samples (p=0.012; Table 9).

eCPMV Immunotherapy is Associated With Improved Survival in Treated Patients

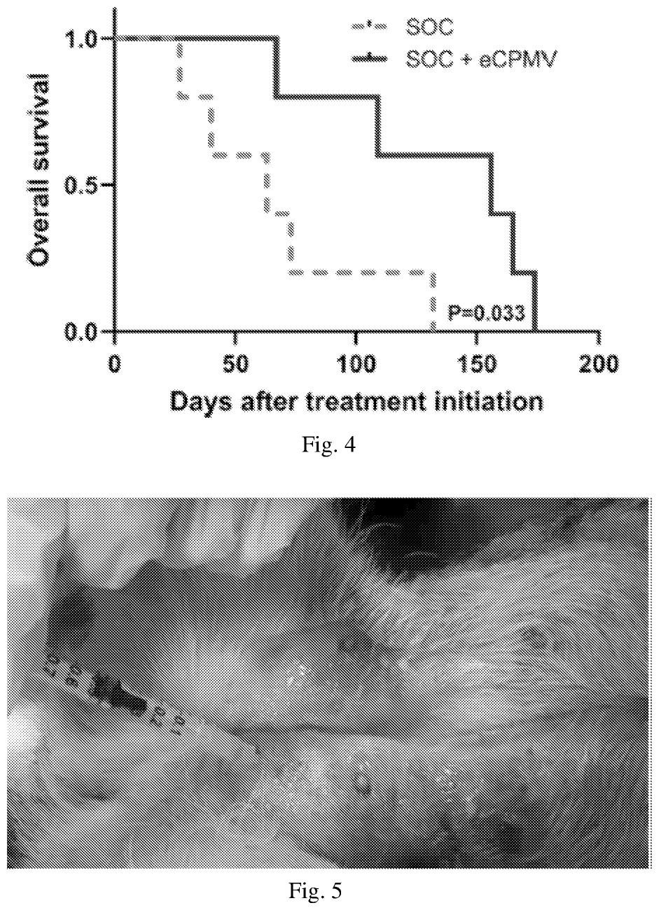

The mean survival time of dogs treated with eCPMV was significantly higher than the mean survival time of the control group which received the medical therapy but not eCPMV immunotherapy (134 days vs 67 days (data not shown); FIG. 4, p=0.033). All patients, except P1 (who died of unrelated renal failure), were euthanized due to tumor progression.

This Example established the potential clinical utility of eCPMV nanoparticles as a novel immunotherapy against canine IMC, a highly aggressive and metastatic disease. Given the extensive local involvement, the presence of coagulopathies and distant metastatic disease, surgery is not recommended for IMC.

The translational value of our findings relates to tumor regression in response to eCPMV immunotherapy. All IMC patients enrolled in this study who received just two injections of eCPMV immunotherapy within a ˜2-week period had tumor regression. The positive response to eCMPV therapy translated into a survival of ˜6 months for patient P1 who died of renal failure with no evidence of metastatic disease in the kidneys. Necropsy analysis showed that renal failure was probably caused by COX inhibitors and kidney infarcts, which could be associated with tumor-related coagulopathies. Further, overall survival benefit was statistically significant in IMC patients treated with eCMPV therapy compared with IMC control patients who lived an average of 1 month without treatment. Although an excellent survival of ˜6 months was reported for IMC patients receiving more aggressive medical therapy (piroxicam, thalidomide, and toceranib) plus radiation therapy, to our knowledge, reports of improved survival in IMC patients receiving medical therapy and just a single agent as demonstrated in this study are very limited. Collectively, our results support the beneficial effect of eCPMV immunotherapy on survival in companion dogs diagnosed with spontaneous IMC, as previously reported in metastatic canine oral melanoma tumors.

Although the blood analysis in eCPMV-treated IMC dogs demonstrated individual fluctuations in the levels of lymphocytes, monocytes, mature and immature neutrophils, immature neutrophils always remained above the pretreatment levels. Similar fluctuations within normal ranges were observed in blood biochemistry analysis. Hence, eCPMV immunotherapy does not induce toxic events. Of note, of the 13 cytokines analyzed, IL-8 was the only cytokine showing notable changes in the blood of eCPMV-treated IMC patients. IL-8 is secreted by blood monocytes, alveolar macrophages, fibroblasts, endothelial cells, and epithelial cells. The pleiotropic functions of IL-8 include varied effects such as recruitment of neutrophils, stimulation of angiogenesis and stimulation of tumor-cell proliferation. While high systemic IL-8 levels are associated with adverse cancer prognosis, resistance to immunotherapy in human tumors, and worse outcome in canine mammary tumors, changes in plasma IL-8 levels did not correlate with response in eCPMV-treated IMC dogs. These findings suggest a different role of IL-8 in the biology of IMC patients or in the response to a potent immunogenic agent such as eCPMV nanoparticles.

In previous syngeneic murine tumor models, including the breast 4T1 model, we demonstrated that eCPMV particles were rapidly taken up by and activate neutrophils in the tumor microenvironment (TME) as an important part of the antitumor immune response. In this canine model, we observed that eCPMV immunotherapy induced an increase in blood mature and immature blood neutrophils, transient increases in IL-8 in the blood, and a strong neutrophilic infiltration in the tumor mass. Of note, neutrophil infiltration was predictive of treatment benefit in murine mastocytomas treated with intratumoral injection of Complete Freund's adjuvant (CFA, containing killed mycobacteria). Mycobacteria triggers an immune response by activating toll-like receptors (TLR) 2 and 4. Furthermore, intratumoral CFA injections resulted in high neutrophil influx accompanied by extensive tumor necrosis in human renal, prostate, bladder and cervical carcinomas; and an increased B and T cell infiltration was observed in treated canine mastocytomas, providing evidence of a systemic immune response to the in situ treatment similar to what we observed in our eCPMV-treated dogs.

We have previously demonstrated that eCPMV induced immune response in mice by activating TLR2 and TLR4 to generate a high level inflammatory response. This leads to phagocyte activation and switching their immune suppressive status to become antigen presenting cells carrying tumor antigens to the draining lymph nodes where effector T cells are generated. These activated T cells, along with the activated myeloid cells, are responsible for the anti-tumor response and systemic effects which result in elimination of circulating and distant tumor deposits. While we cannot be sure that details of responses to eCPMV do not vary between mice and dogs, it is reasonable to assume that they are quite similar overall.

The lower Ki-67 PI of cancer cells and the absence of an increase in the percentage of tumor cells undergoing apoptosis suggest that apoptosis is not the mechanism of cell death, and that eCPMV-activated neutrophils are potentially responsible for tumor cell death. Importantly, the fact that post-treatment tumor emboli show strong neutrophilic activity and similar staining of MPO, Ki-67, and CC-3 as in post-treatment tumor samples is of clinical relevance. Tumor emboli in human IBC are associated with metastatic behavior of IBC. Induction of cell killing in tumor emboli could explain delayed metastatic formation and better survival in eCPMV-treated patients.

Blood samples from canine cancer patients have an increased Treg⁺/CD8⁺ T cell ratio compared with healthy dogs and a higher ratio was associated with short survival in canine osteosarcoma. Our analysis demonstrated a steady decrease of the peripheral blood Treg⁺/CD8⁺ ratio in all dogs during treatment with a sharp increase observed in both surgically treated dogs (P1 and P5) after surgery. Although both metronomic cyclophosphamide and toceranib decrease Treg in dogs, we consider that eCPMV immunotherapy was the driving factor in decreasing the Treg⁺/CD8⁺ ratio because it was the only therapy given to all dogs for 2 weeks. Further, during the time the patient was not undergoing treatment, a sharp increase in the Treg⁺/CD8⁺ ratio in patient P1 was observed. At that time, the patient was on anti-COX-2 and cyclophosphamide therapy without eCPMV immunotherapy. The post-surgery sharp increase in Treg⁺/CD8⁺ ratio in patients P1 and P5 is likely surgery related. Of note, a post-treatment decrease in FoxP3⁺/CD3⁺ ratio was also observed in P1, P2, and P3 tumor samples (Table 3), indirectly indicating a decrease in immunosuppressive Treg lymphocytes. Our results extend previous findings that CPMV therapy induced Treg depletion in 4T1 BC model.

Changes in CD8⁺GZMB⁺ T cells in peripheral blood induced by eCPMV immunotherapy behave as a lagging biomarker of response to therapy. While the effect is clearly observed in patient P1, the most responsive patient to eCPMV immunotherapy, studies with more patients are needed to draw definitive conclusions.

Efficacious therapies combining treatments like chemotherapy with radiation therapy are associated with adverse events like cutaneous, hematologic, and gastrointestinal toxicities in dogs. Blood plasma biochemistry studies did not show abnormal changes in any of the indicators used to track potential adverse events caused by eCMPV therapy. In agreement with previous studies, we did not observe adverse reactions at the injection site or systemic reactions after any eCPMV administration, and QOL was even improved in three out of five patients. Hence, eCMPV therapy appears to be safe and well tolerated.

The exceedingly high efficacy of neoadjuvant in situ eCPMV vaccination in IMC patients opens the possibility of employing in situ eCPMV vaccination as a potential novel and effective neoadjuvant immunotherapy against IBC, a therapeutically orphan disease with poor outcome.

Most canine tumors express PD-L1 constitutively and both innate and adaptive immune stimuli can further upregulate PD-L1 expression. We expect that combining in situ eCPMV immunotherapy with cross-functional human anti-PD-L1 inhibitors or canine anti-PD-1/PD-L1 inhibitors, as they become available, will result in higher efficacy than single agent used as monotherapy. We have demonstrated that in situ eCPMV synergizes with systemic checkpoint immunotherapy in various mouse models.

Our findings support the implementation of neoadjuvant in situ eCPMV immunotherapy as a novel and safe immunotherapy against IMC and suggest that neutrophils are drivers of tumor cell death. We envision neoadjuvant in situ eCPMV immunotherapy as a treatment for IBC patients.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. A method of treating cancer in a subject in need thereof, the method comprising:
administering to the subject a neoadjuvant therapy that includes an in situ vaccine and optionally an immune check point therapy prior to surgical resection and/or radiotherapy of the cancer, the in situ vaccine comprising at least one of cowpea mosaic virus or cowpea mosaic virus-like particles;
treating the cancer by surgical resection and/or radiotherapy; and
optionally administering an adjuvant therapy to the subject after surgical resection and/or radiotherapy of the cancer.

2. The method of claim 1, wherein the cancer is a locally advanced, metastatic, and/or inflammatory cancer.

3. The method of claim 1, wherein the cancer is inflammatory breast cancer.

4. The method of claim 1, wherein the cancer is stage III or stage IV inflammatory breast cancer.

5. The method of claim 1, wherein the cancer is PD-L1+ inflammatory breast cancer.

6. The method of claim 1, wherein the neoadjuvant is administered in the absence of chemotherapy.

7. The method claim 1, wherein the immune check point therapy includes administration of an immune check point inhibitor to the subject.

8. The method of claim 1, wherein the immune check point therapy includes administration of at least one of an anti-PD-1 antibody or an anti-PD-L1 antibody to the subject.

9. The method of claim 1, wherein the adjuvant therapy comprises at least one of radiotherapy, immunotherapy, chemotherapy, ultrasound therapy, or hormone therapy.

10. The method of claim 1, wherein the neoadjuvant therapy is administered in an amount effective to reduce cancer burden in the subject.

11. The method of claim 1, wherein the neoadjuvant therapy is administered in an amount effective to decrease Treg/CD8+ ratio and/or increase CD8+GZMB+ T cell levels in blood of the subject.

12. The method of claim 1, wherein cancer is inflammatory breast cancer, which is inoperable prior to administration of the neoadjuvant therapy and optional immune check point therapy, and the neoadjuvant therapy and optional immune checkpoint therapy is administered at an amount effective for the inflammatory breast cancer to be operable by surgical resection.

13. The method of claim 1, wherein the cowpea mosaic virus and cowpea mosaic virus-like are administered by injection into the cancer.

14. A method of treating breast or mammary cancer in a subject in need thereof, the method comprising:
administering to a subject with locally advanced or inflammatory breast or mammary cancer a neoadjuvant therapy that includes an in situ vaccine and optionally an immune check point therapy prior to surgical resection of the breast or mammary cancer, the in situ vaccine comprising at least one of cowpea mosaic virus or cowpea mosaic virus-like particles;
removing the breast or mammary cancer by surgical resection; and
optionally administering an adjuvant therapy to the subject after surgical resection of the breast or mammary cancer.

15. The method of claim 14, wherein the breast cancer is inflammatory breast cancer.

16. The method of claim 14, wherein the inflammatory breast cancer is stage III or stage IV inflammatory breast cancer.

17. The method of claim 14, wherein the inflammatory breast cancer is PD-L1+ inflammatory breast cancer.

18. The method of claim 14, wherein the neoadjuvant is administered in the absence of chemotherapy.

19. The method of claim 14, wherein the immune check point therapy includes administration of an immune check point inhibitor to the subject.

20. The method of claim 14, wherein the immune check point therapy includes systemic administration of at least one of an anti-PD-1 antibody or an anti-PD-L1 antibody to the subject.

21-36. (canceled)
